SEQUENCE LISTING (23 sequences)

SEQ ID NO: 1 - protein, 716 aa, adeno-associated virus po1
MSFVDHPPDWLEEIGEGLKEFLGLEPGPPKPKPNQQKQDDARGLVLPGYNYLGPGNGLDRGEPVNRADEVAREHDISYNEQLQAGDNPYLKYNHADAEFQEKLADDTSFGGNLGKAVFQAKKRVLEPFGLVEEPVKTAAKGERIDDHYPKKKKARIEETEAGTSGAQQLQIPAQPASSLGADTMSAGGGSPLGDNNQGADGVGNASGDWHCDSTWMGDRVITKSTRTWVLPSYNNHQYLEIHSGSVDGSNANAYFGYSTPWGYFDFNRFHSHWSPRDWQRLVNNYWGFRPRSLKVKIFNIQVKEVTTQDGTTTIANNLTSTVQVFTDNDYQLPYVIGNGTEGCLPAFPPQVFTLPQYGYATLNRNNTDDPTERSSFFCLEYFPSKMLRTGNNFEFTYSFEEVPFHCSFAPSQNLFKLANPLVDQYLYRFVSTDTSGNLQFQKNLKARYANTYKNWFPGPMCRTQGWYTSAGTYNNKGVANFDTSNKMELEGASYQVNPQPNGMTNTLQDSNKYALENTMIFNAQNAPPGTTSLYQENNLLITSESETQPVNRLAYNTGGQVSNNNQNSNTHPTVGVYNHQEVLPGSVWMDRDVYLQGPIWAKIPETGAHFHPSPAMGGFGLKHPPPMMLIKNTPVPSNVAAFSDVPVKSFITQYSTGQVTVEIEWELKKENSKRWNPEIQYTNNYNNPTFVDFAPDTSGEYRTTRAIGTRYLTRPL

SEQ ID NO: 2 - protein, 7 aa, artificial sequence
  synthetic peptide (A1)
MPLGAAG

SEQ ID NO: 3 - protein, 9 aa, artificial sequence
  synthetic peptide
GMPLGAAGA

SEQ ID NO: 4 - protein, 15 aa, artificial sequence
  synthetic peptide
GQRGMPLGAAGAQAA

SEQ ID NO: 5 - protein, 725 aa, artificial sequence
  synthetic polypeptide (AAVpo1A1 capsid protein)
MSFVDHPPDWLEEIGEGLKEFLGLEPGPPKPKPNQQKQDDARGLVLPGYNYLGPGNGLDRGEPVNRADEVAREHDISYNEQLQAGDNPYLKYNHADAEFQEKLADDTSFGGNLGKAVFQAKKRVLEPFGLVEEPVKTAAKGERIDDHYPKKKKARIEETEAGTSGAQQLQIPAQPASSLGADTMSAGGGSPLGDNNQGADGVGNASGDWHCDSTWMGDRVITKSTRTWVLPSYNNHQYLEIHSGSVDGSNANAYFGYSTPWGYFDFNRFHSHWSPRDWQRLVNNYWGFRPRSLKVKIFNIQVKEVTTQDGTTTIANNLTSTVQVFTDNDYQLPYVIGNGTEGCLPAFPPQVFTLPQYGYATLNRNNTDDPTERSSFFCLEYFPSKMLRTGNNFEFTYSFEEVPFHCSFAPSQNLFKLANPLVDQYLYRFVSTDTSGNLQFQKNLKARYANTYKNWFPGPMCRTQGWYTSAGTYNNKGVANFDTSNKMELEGASYQVNPQPNGMTNTLQDSNKYALENTMIFNAQNAPPGTTSLYQENNLLITSESETQPVNRLAYNTGGQVSNNNQGQRGMPLGAAGAQAATVGVYNHQEVLPGSVWMDRDVYLQGPIWAKIPETGAHFHPSPAMGGFGLKHPPPMMLIKNTPVPSNVAAFSDVPVKSFITQYSTGQVTVEIEWELKKENSKRWNPEIQYTNNYNNPTFVDFAPDTSGEYRTTRAIGTRYLTRPL

SEQ ID NO: 6 - DNA, 21 bp, artificial sequence
  synthetic oligonucleotide
aaaacgagcagtgacgtgagc

SEQ ID NO: 7 - DNA, 20 bp, artificial sequence
  synthetic oligonucleotide
ttcagtcatgctgctagcgc

SEQ ID NO: 8 - DNA, 25 bp, artificial sequence
  synthetic oligonucleotide
tgcacggaagcgtctcgtctcagtc

SEQ ID NO: 9 - DNA, 25 bp, artificial sequence
  synthetic oligonucleotide
ttggttgtccagtttggagtctact

SEQ ID NO: 10 - DNA, 20 bp, artificial sequence
  synthetic oligonucloetide
ccgtcactgcaatgcacaag

SEQ ID NO: 11 - DNA, 19 bp, artificial sequence
  synthetic oligonucleotide
atatcaagctcgttttgac

SEQ ID NO: 12 - protein, 7 aa, artificial sequence
  synthetic peptide
  VARIANT (2)..(2): X is any amino acid
  VARIANT (3)..(3): X is any amino acid
  VARIANT (5)..(5): X is any amino acid
  VARIANT (6)..(6): X is any amino acid
  VARIANT (7)..(7): X is any amino acid
NXXRXXX

SEQ ID NO: 13 - DNA, 2178 bp, artificial sequence
  synthetic polynucleotide encoding AAVpo1A1 capsid (VP1) protein
  CDS (1)..(2178)
atgtcgtttgttgatcaccctccagattggcttgaggagattggtgagggtctaaaggagtttttgggactcgaacctggcccacccaaaccgaagcccaaccagcagaagcaagacgacgcccgtggtcttgtactgcctggatataattacctgggacccggaaacggtctcgaccgcggagaacctgtcaaccgggctgacgaggtcgcgcgagagcacgacatctcgtacaacgagcagctccaggcgggagacaacccctacctcaagtacaaccacgcggacgccgagtttcaggagaagctcgcggacgacacctccttcgggggcaacctcggcaaggcagtctttcaggccaaaaaaagggttctcgaaccttttggcctggttgaggagcctgttaagacggctgctaaaggcgagcggatagacgaccactatcccaaaaagaagaaggctcggatcgaagagaccgaagctggaaccagcggagcccagcagctgcagatcccagcccaaccagcctcaagtttgggagctgatacaatgtctgcgggaggtggcagcccactgggcgacaataaccaaggcgccgatggagtgggcaatgcctcgggagattggcattgcgattccacgtggatgggggaccgagtcatcaccaagtccacccgaacctgggtgctgcccagctacaacaaccatcagtaccttgagatccacagcggttccgtcgacggaagcaacgctaacgcttattttggatacagcaccccctgggggtactttgacttcaaccgcttccacagccactggagcccccgagactggcagcgactcgtcaacaactactggggattcagaccccggtccctcaaggtcaagatctttaacatccaagtcaaggaagtcacgacgcaggacggcaccaccaccatcgccaacaacctcacctccaccgtccaagtgtttacggacaacgactaccagctaccgtacgtcatcggcaacggaacggaggggtgcctgccggccttccctccgcaggtctttacgctgccgcagtacggctacgcgacactgaaccgtaacaacaccgacgatcccaccgagcggagcagtttcttctgcctggaatactttcccagcaagatgctgcggacgggcaacaactttgaattcacctacagcttcgaggaggtgcccttccactgcagcttcgctcccagccagaacctcttcaagctggccaatccgctggtggaccagtacctgtaccgctttgtgagcaccgacacttccggtaacctacagttccaaaagaacttgaaggccagatatgccaacacttacaagaattggtttccggggcccatgtgccggacccagggctggtacacaagcgcgggcacatataacaacaaaggcgttgccaactttgatacttcaaacaagatggaactggagggggctagttaccaagtaaaccctcaaccaaatggaatgacaaacacgcttcaggatagtaacaaatacgcgcttgaaaacaccatgatcttcaacgcacagaacgcccctccgggaacgacctctctgtaccaggagaacaatcttttgataaccagcgagagcgagacgcagcctgtgaaccgattggcctacaacaccggtggtcaggtatcaaacaacaaccagggccagagaggcatgccattaggagcggcaggcgcccaggcggccacggtcggagtatacaatcaccaggaagtgttgcctggtagcgtgtggatggacagagacgtataccttcagggccccatctgggccaaaatcccggagacaggggcacactttcatccttctccggctatgggcggattcggactcaaacacccaccgcccatgatgctcatcaagaacacaccggtacctagcaacgtcgctgccttctctgacgtgcccgttaagagcttcatcacccagtacagcaccggacaggtcacggtggagattgaatgggagctcaagaaagaaaactccaagaggtggaatcccgagatacagtacaccaacaactacaacaaccctacattcgtggactttgctccagacacctccggcgagtacaggactacgagggctattggaacccgttaccttacccgacccctgtaa

SEQ ID NO: 14 - protein, 725 aa, artificial sequence; identical to SEQ ID NO: 5
  Synthetic Construct

SEQ ID NO: 15 - DNA, 20 bp, artificial sequence
  synthetic oligonucleotide (primer SMN1-FW)
cagtgcaggctgcctatcag

SEQ ID NO: 16 - DNA, 18 bp, artificial sequence
  synthetic oligonucleotide (primer SMN1-RV)
tgtgggccagggcattag

SEQ ID NO: 17 - DNA, 17 bp, artificial sequence
  synthetic oligonucleotide (proble SMN1)
aagtggtggctggtgtg

SEQ ID NO: 18 - DNA, 20 bp, artificial sequence
  synthetic oligonucleotide (primer SMN1-FW)
gctgcctccatttccttctg

SEQ ID NO: 19 - DNA, 24 bp, artificial sequence
  synthetic oligonucleotide (primer SMN1-RV)
acatacttcccaaagcatcagcat

SEQ ID NO: 20 - DNA, 30 bp, artificial sequence
  synthetic oligonucleotide (probe SMN1)
caccacctcccatatgtccagattctcttg

SEQ ID NO: 21 - DNA, 20 bp, artificial sequence
  synthetic oligonucleotide (primer RPLP0-FW)
ctctggagaaactgctgcct

SEQ ID NO: 22 - DNA, 23 bp, artificial sequence
  synthetic oligonucleotide (primer RPLP0-RV)
ctgcacatcactcagaatttcaa

SEQ ID NO: 23 - DNA, 27 bp, artificial sequence
  synthetic oligonucleotide (probe RPLP0)
aggacctcactgagattcgggatatgc

(19) United States
(12) Patent Application Publication
(10) Pub. No.: US 2023/0173102 A1
(43) Pub. Date: Jun. 8, 2023

(54) Use of a Synthetic AAV Capsid for Gene Therapy of Muscle and Central Nervous System Disorders

(71) Applicants: Genethon, Evry-Courcouronnes (FR); Universität Heidelberg, Heidelberg (DE); Universite d'Evry val d'Essonne, Evry-Courcouronnes (FR); Institut National de la Santé et de la Recherche Médicale, Paris (FR)

(72) Inventors: Ana Buj Bello, Paris (FR); Edith Renaud-Gabardos, Saint Fargeau-Ponthierry (FR); Dirk Grimm, Heidelberg (DE); Jonas Weinmann, Heidelberg (DE)

(73) Assignees: Genethon, Evry-Courcouronnes (FR); Universität Heidelberg, Heidelberg (DE); Universite d'Evry val d'Essonne, Evry-Courcouronnes (FR); Institut National de la Santé et de la Recherche Médicale, Paris (FR)

(21) Appl. No.: 17/921,727

(22) PCT Filed: Apr. 28, 2021
(86) PCT No.: PCT/EP2021/061204

§ 371 Date: Oct. 27, 2022

(30) Foreign Application Priority Data
Apr. 28, 2020 (EP) 20305412.7

(51) Int. Cl. A61K 48/00; A61P 21/00

(52) U.S. Cl. CPC A61K 48/0058; A61K 48/0075; A61P 21/00

(57) ABSTRACT

The invention relates to the use of a recombinant porcine adeno-associated virus (AAV) vector comprising a peptide-modified porcine AAV serotype 1 (AAVpol) capsid in gene therapy of muscle and/or central nervous system (CNS) disorders, in particular neuromuscular diseases such as genetic neuromuscular diseases.

### FIELD OF THE INVENTION

The invention relates to the use of a recombinant porcine adeno-associated virus (AAV) vector comprising a peptide-modified porcine AAV serotype 1 (AAVpo1) capsid in the gene therapy of muscle and/or central nervous system (CNS) disorders, in particular neuromuscular diseases such as genetic neuromuscular diseases.

### BACKGROUND OF THE INVENTION

Recombinant Adeno-Associated Virus (rAAV or AAV) vectors are widely used for in vivo gene transfer and clinical trials using AAV vectors are currently taking place for the treatment of a number of diseases.

AAV vectors are non-enveloped vectors composed of a capsid of 20 nm of diameter and a single strand DNA of 4.7 kb. The genome carries two genes, rep and cap, flanked by two palindromic regions named Inverted terminal Repeats (ITR). The cap gene codes for three structural proteins VP1, VP2 and VP3 that compose the AAV capsid. VP1, VP2 and VP3 share the same C-terminal end which is all of VP3. Using AAV2 has a reference, VP1 has a 735 amino acid sequence (GenBank YP_680426); VP2 (598 amino acids) starts at the Threonine 138 (T138) and VP3 (533 amino acids) starts at the methionine 203 (M203).

Tissue specificity is determined by the capsid serotype and commonly used AAV serotypes isolated from human (AAV2, 3, 5, 6) and non-human primates (AAV1, 4, 7-11) can transduce specific organs more efficiently than others, such as AAV6, AAV8, AAV9 and AAV-rh74 in muscle tissue and AAV2, AAV9, AAVrh10, AAVcy.10, AAV-PHP.B, AAV-PHP.EB and clade F AAVHSC such as AAVHSC7, AAVHSC15 and AAVHSC17 in nervous tissue.

However, all commonly used naturally occurring AAV serotypes and variants thereof tested to date have a propensity to accumulate within the liver. This causes problems, in particular when the AAV vector is administered by the systemic route. Firstly, a transgene aimed to be expressed in muscle may have toxic effects on the liver. Secondly, AAV vector entry in liver reduces the amount of vector available for muscle or nervous tissue. Consequently, higher doses of AAV vectors are required. This increases the possibility to induce liver toxicity and the cost of vector production.

In addition, pre-existing immunity to AAV is a drawback of commonly used AAV vector serotypes isolated from human and non-human primates, in particular AAV2 which is seroprevalent in up to 80% of the human population, as well as other serotypes (Fu et al., Hum Gene Ther Clin Dev., 2017 Dec;28(4):187-196; Stanford et al., Res Pract Thromb Haemost., 2019, 3: 261-267°.

Recombinant AAV vectors have been generated using capsids from different porcine AAVs (AAVpo1, po2.1, po4 to 6) and following systemic administration in mice, strong transgene expression in all major skeletal muscle types combined with poor transduction of other tissues including complete detargeting from the liver was reported for AAVpo1. AAV po2.1 was also detargeted from the liver after peripheral administration, whereas AAVpo4 and AAVpo6 efficiently transduced all the major organs sampled including the brain. The porcine AAV vectors were not cross-neutralized by antisera generated against all other commonly used AAVs or pooled human Igs (Bello et al., Gene Therapy, 2009, 16, 1320-1328. doi: 10.1038/gt.2009.82; Bello et al., Sci Rep., 2014, 4, 6644, DOI: 10.1038/srep06644; Tulalamba et al., Gene Therapy, 2019, doi.org/10.1038/s41434-019-0106-3; Puppo et al., PLOS ONE, 2013, 8, e59025; WO 2009/030025). Altogether, this makes recombinant porcine AAV vectors, in particular AAVpo1 and AAV po2.1 that are detargeted from the liver, attractive vectors for human gene therapy of muscle diseases. However, the transgene expression levels in muscle achieved with recombinant porcine AAV vectors although high, were still within a twofold to threefold range lower than that of AAV9, often considered as the gold standard for muscle-directed gene therapies. In addition, the transduction efficiency of the brain reported with AAVpo1 and AAV po2.1 was low.

Libraries of AAV capsid variants displaying short peptides on the surface of various AAV serotypes have been generated to screen for gene therapy vectors with altered cell specificites and/or transduction efficiencies (Börner et al., Molecular Therapy, April 2020, 28, 1017-1032; Kienle EC (Dissertation for the degree of Doctor of natural Sciences, Combined Faculties for the Natural Sciences and for Mathematics of the Ruperto-Carola University of Heidelberg, Germany, 2014; WO 2018/189244). Peptide-modified AAV1, 7-9, rh10 and DJ capsids effective in in vitro transduction of human T cell lines, primary human macrophages, hepatocytes, astrocytes were reported. Peptides sharing the motif NXXRXXX (SEQ ID NO: 12) were disclosed as more performant for the transduction of numerous cell types in vitro in the context of multiple AAV serotypes

The ability to transduce different muscle groups and the central nervous system efficiently; selectively and safely with systemically-delivered AAV vectors would be beneficial for gene therapy of many human diseases.

### SUMMARY OF THE INVENTION

The inventors have used a recombinant porcine adeno-associated virus (AAV) vector comprising a peptide-modified capsid protein from porcine AAV serotype 1 (AAVpo1) to deliver a therapeutic gene of interest by systemic administration in mice. Some of the best AAV vector serotypes commonly used for muscle (AAV8, AAV9) or CNS (AAV9, AAVrh10) transduction were tested at the same time for comparison. The inventors have found that, surprisingly, the peptide-modified AAVpo1 vector advantageously achieved transgene expression levels in different muscle groups and in the central nervous system (brain and spinal cord) that were at least equivalent if not superior to that of AAV9 vector while at the same time being detargeted from the liver. Furthermore, it is expected that this porcine AAV vector will not be neutralised by pre-existing antibodies to common AAV (human and non-human primates AAV). For all these reasons, the use of such peptide-modified AAVpo1 vector represents an efficient, selective and potentially safer therapeutic approach for gene therapy of muscle and/or CNS disorders, in particular neuromuscular diseases such as genetic neuromuscular diseases. In some embodiments, the peptide-modified AAVpo1 vector is used to target nervous system cells or nervous system cells and muscle cells for treating nervous system diseases and neuromuscular diseases, in particular genetic nervous system diseases and genetic neuromuscular diseases.

Therefore, one aspect of the invention relates to a recombinant porcine adeno-associated virus (AAV) vector comprising a peptide-modified capsid protein from porcine AAV serotype 1 for the gene therapy of muscle and central nervous system (CNS) disorders; in particular nervous system disorders and neuromuscular disorders affecting the nervous system, such as CNS disorders and neuromuscular disorders affecting the CNS

In some embodiments, the recombinant porcine AAV vector for use according to the invention is characterized by the combination of liver detargeting and transgene expression levels in different muscle groups, and in the brain and spinal cord that are at least equivalent if not superior to that of AAV9 vector, after systemic administration, in particular intravenous administration.

In some embodiments, the peptide-modified capsid protein comprises at least one peptide comprising the sequence MPLGAAG (SEQ ID NO: 2) or a variant comprising only one or two amino acid mutations (insertion, deletion, substitution) in said sequence, preferably one or two amino acid substitutions in said sequence. In some preferred embodiments, the peptide comprises the sequence GMPLGAAGA (SEQ ID NO: 3), or a variant comprising up to four (1, 2, 3 or 4) amino acid mutations (insertion, deletion, substitution) in said sequence, preferably only one or two amino acid deletions or substitutions in said sequence, said deletions being advantageously at the N- and/or C-terminal ends. In some preferred embodiments, the sequence SEQ ID NO: 2 or 3 or variant thereof is flanked by up to five amino acids at its N- and/or C-terminal ends, such as GQR and GAA, respectively at its N-and C-terminal ends. In some more preferred embodiments, the peptide comprises or consists of the sequence GQRGMPLGAAGAQAA (SEQ ID NO: 4).

In some embodiments, the peptide is inserted between residues N567 and S568 or between residues N569 and T570 of the capsid protein; said positions being determined by alignment with SEQ ID NO: 1. Preferably, the peptide is inserted between positions N567 and S568 and replaces all the residues from positions 565-567 and 568-570 or the peptide is inserted between positions N569 and T570 and replaces all the residues from positions from positions 567-569 and 570-572; said positions being determined by alignment with SEQ ID NO: 1.

In some preferred embodiments, said peptide-modified AAVpo1 capsid protein comprises a sequence selected from the group consisting of the sequence SEQ ID NO: 5 and the sequences having at least 95%, 96 % 97%, 98% or 99% identity with SEQ ID NO: 5 which comprise said peptide according to the present disclosure, and the fragment thereof corresponding to VP2 or VP3 capsid protein.

In some embodiments, the recombinant porcine AAV vector is a vector particle packaging a gene of interest for therapy.

In some preferred embodiments, the gene of interest is operably linked to a promoter functional in neurons and/or glial cells.

In some preferred embodiments, the gene of interest for therapy is selected from the group consisting of:
(i) therapeutic genes;
(ii) genes encoding therapeutic proteins or peptides such as therapeutic antibodies or antibody fragments and genome editing enzymes; and
(iii) genes encoding therapeutic RNAs such as interfering RNAs, guide RNAs for genome editing and antisense RNAs capable of exon skipping.

In some embodiments, the disease is a neuromuscular disease, preferably a genetic neuromuscular disease. Preferably, the disease is a neuromuscular disease affecting the nervous system, preferably a genetic neuromuscular disease affecting the nervous system.

In some embodiments, the genetic neuromuscular disease is selected from the group comprising: (i) myopathies, such as hereditary cardiomyopathies, metabolic myopathies, other myopathies, distal myopathies, muscular dystrophies and congenital myopathies; and (ii) spinal muscular atrophies (SMAs) and motor neuron diseases; preferably congenital myopathies and muscular dystrophies, and spinal muscular atrophies (SMAs) and motor neuron diseases.

In some embodiments, the gene of interest for therapy is a functional version of a gene responsible for a genetic neuromuscular disorder selected from the group comprising: Duchenne muscular dystrophy, Becker muscular dystrophy, Limb-girdle muscular dystrophies, Myotonic dystrophy, Myotubular myopathy, Centronuclear myopathies, Nemaline myopathies, Selenoprotein N-related myopathy, Pompe disease, Glycogen storage disease III, Spinal muscular atrophy, Amyotrophic lateral sclerosis, or a therapeutic RNA targeting said gene responsible for the disease.

In some embodiments, said gene responsible for the genetic neuromuscular disorder is selected from the group comprising: DMD, CAPN3, DYSF, FKRP, ANO5, MTM1, DNM2, BIN1, ACTA1, KLHL40, KLHL41, KBTBD13, TPM3, TPM2, TNNT1, CFL2, LMOD3, SEPN1, GAA, AGL, SMN1, and ASAH1 genes.

In some preferred embodiments, the genetic neuromuscular disease is selected from the group comprising : (i) myopathies, such as muscular dystrophies including congenital muscular dystrophies ; (ii) spinal muscular atrophies (SMAs) and motor neuron diseases; (iii) Myotonic syndrome, in particular myotonic dystrophy type 1 and type 2; (iv) Hereditary motor and sensory neuropathies; (v) Hereditary paraplegia and Hereditary ataxia; (vi) Congenital myasthenic syndromes ; preferably Congenital myasthenic syndromes, muscular dystrophies including congenital muscular dystrophies, and spinal muscular atrophies (SMAs) and motor neuron diseases.

In some preferred embodiments, the gene of interest for therapy is a functional version of a gene responsible for a genetic neuromuscular disorder affecting the nervous system selected from the group comprising: Duchenne muscular dystrophy and Becker muscular dystrophy (DMD gene), Limb-girdle muscular dystrophies (DYSF, FKRP genes), Myotonic dystrophy type 1 (DMPK gene) and type 2 (CNBP/ZNF9 gene), Centronuclear myopathies (DNM2, BIN1 genes), Pompe disease (GAA gene), Glycogen storage disease III (AGL gene), Spinal muscular atrophy (SMN1, ASAH1 genes), Amyotrophic lateral sclerosis (SOD1, ALS2, SETX, FUS, ANG, TARDBP, FIG4, OPTN and others), Hereditary paraplegia (SPAST (SPG4), SPG7, and other SPG genes such as SPG11, SPG20 and SPG21), Charcot-Marie-Tooth, Type 4B1 (MTMR2) and Congenital myasthenic syndromes (CHAT, AGRN), or a therapeutic RNA targeting said gene responsible for the disease.

In some more preferred embodiments, said gene responsible for the genetic neuromuscular disorder affecting the nervous system is selected from the group comprising: DMD, DYSF, FKRP, DNM2, BIN1, GAA, AGL, SMN1 and ASAH1 genes.

In other more preferred embodiments, said gene responsible for the genetic neuromuscular disorder affecting the nervous system is selected from the group comprising: FKTN, POMT1, POMT2, POMGNT1, POMGNT2, LMNA, ISPD, GMPPB, LARGE, LAMA2, TRIM32, and B3GALNT2 genes.

In some embodiments, the recombinant porcine AAV vector is for use in the gene therapy of Spinal muscular atrophy, and said vector comprising a peptide-modified capsid protein comprising the sequence SEQ ID NO: 5 or a sequence having at least 95%, 96%, 97%, 98% or 99% identity with said sequence which comprises the peptide of any one of SEQ ID NO: 2 to 4, said vector further packaging a human SMN1 gene operably linked to a promoter functional in neurons and/or glial cells.

In some embodiments, the recombinant porcine AAV vector according to the disclosure is administered systematically, preferably intravenously.

In some embodiments, the recombinant porcine AAV vector according to the disclosure is used in a method for the treatment of a neuromuscular disease.

### DETAILED DESCRIPTION OF THE INVENTION

### Peptide-Modified AAVpo1 Vector

The invention relates to a recombinant adeno-associated virus vector comprising a peptide-modified porcine AAV serotype 1 capsid protein for use in the gene therapy of muscle and nervous system disorders, such as muscle and central nervous system (CNS) disorders. The recombinant adeno-associated virus vector comprising a peptide-modified porcine AAV serotype 1 capsid protein may be used in the gene therapy of diseases affecting only the nervous system (PNS and/or CNS), or affecting both the nervous system and muscle. These include in particular CNS diseases and neuromuscular diseases.

The porcine AAV serotype 1 (AAVpo1) vector comprising a peptide-modified capsid protein (or peptide-modified AAVpo1 vector) for the use according to the invention combines detargeting from off-target organ(s) including in particular the liver and high transgene expression levels in target organs (i.e. nervous system, such as CNS; or muscles and nervous system, such as muscles and CNS) following systemic administration.

As used herein, the term “detargeting” refers to the reduction of vector transduction and transgene expression in off-target organs to minimal levels, preferably as close as possible to the limit of detection. The peptide-modified AAVpo1 vector according to the present disclosure which is detargeted at the transduction level advantageously comprises at least 10 fold less vector genome copy number per diploid genome compared to AAV8, AAV9 vector following systemic administration at the same dose. The peptide-modified AAVpo1 vector according to the present disclosure which is detargeted at the level of transgene expression advantageously comprises vector-derived protein levels that are below the endogenous levels of the protein, if using a vector that expresses a human transgene.

As used herein, the term “muscle” refers to cardiac muscle (i.e. heart) and skeletal muscle.

As used herein, the term “muscle cells” refers to myocytes, myotubes, myoblasts, and/or satellite cells.

As used herein, the term “nervous system”, refers to both the central (CNS) and peripheral (PNS) nervous system.

As used herein, the term “central nervous system or CNS” refers to the brain, spinal cord, retina, cochlea, optic nerve, and/or olfactory nerves and epithelium. As used herein, the term CNS cells refer to any cells of the CNS including neurons and glial cells (oligodendrocytes, astrocytes, ependymal cells, microglia).

As used herein, the PNS refers to the nerves and ganglia outside the brain and spinal cord.

As used herein, the term “systemic administration” refers to a route of administration of a substance (vector) into the circulatory system and includes enteral or parenteral administration. Parenteral administration includes injection, infusion, implantation and others.

As used herein, the term “AAV vector” refers to an AAV vector particle.

As used herein, the term “porcine AAV vector or AAVpo1 vector” refers to an AAV vector comprising a porcine AAV serotype 1 capsid protein.

As used herein, the term AAV serotype includes natural and artificial AAV serotypes such as variants and hybrid capsids derived from natural AAV serotypes. AAV serotype refers to a functional AAV capsid which is able to transduce the target organ(s) and express a transgene in said target organ(s).

As used herein, “for use in the gene therapy of muscle and nervous system disorders, such as muscle and central nervous system (CNS) disorders” means “for use in the treatment of muscle and nervous system disorders, such as muscle and central nervous system (CNS) disorders by gene therapy” or “for use in the treatment by gene therapy of muscle and nervous system disorders, such as muscle and central nervous system (CNS) disorders”.

As used herein, “or” means “and/or”.

Neuromuscular disorder (NMD) is a very broad term encompassing a range of conditions that impair the functioning of the muscles, either directly, being pathologies of the voluntary muscle, or indirectly, being pathologies of the peripheral nervous system or neuromuscular junctions. Neuromuscular diseases are a broadly defined group of disorders that all involve injury or dysfunction of peripheral nerves or muscle or neuromuscular junctions. The site of injury can be in the cell bodies (i.e., amyotrophic lateral sclerosis [ALS] or sensory ganglionopathies), axons (i.e., axonal peripheral neuropathies or brachial plexopathies), Schwann cells (i.e., chronic inflammatory demyelinating polyradiculoneuropathy), neuromuscular junction (i.e., myasthenia gravis or Lambert-Eaton myasthenic syndrome), muscle (i.e., inflammatory myopathy or muscular dystrophy), or any combination of these sites. Some neuromuscular diseases are also associated with central nervous system disease, such as ALS.

As used herein “neuromuscular disease or disorder affecting the nervous system” refers to neuromuscular disease comprising a nervous system injury. The neuromuscular disease may further comprise a muscle injury, such as for example a secondary muscle injury as a result of the primary nervous system injury.

In some embodiments, the peptide-modified AAVpo1 vector for the use according to the invention produces high transgene expression levels in target organs (i.e. nervous system, such as CNS; or muscles and nervous system, such as muscles and CNS) following systemic administration, while being at the same time detargeted from the liver. Transduction (vector copy number) in nervous system, such as CNS; or muscle(s) and nervous system, such as muscle(s) and CNS is advantageously increased with the peptide-modified AAVpo1 vector compared to control AAVpo1 vector comprising a capsid not modified by the peptide. Transgene expression level in muscle(s) and nervous system, such as muscle(s) and CNS with the peptide-modified AAVpo1 vector is preferably increased by at least two folds, preferably 3, 4, 5 folds or more in muscle(s), in particular in skeletal muscle(s) and in the central nervous system compared to control AAVpo1 vector comprising a capsid not modified by the peptide. The transgene expression levels achieved with the peptide-modified AAVpo1 vector in different muscle(s) types and in the nervous system, such as in different muscle(s) types and in the CNS is preferably at least of the same magnitude (less than 1.5 fold lower; i.e equivalent to) as that of AAV8, AAV9, AAVrh10 vectors.

Vector transduction and transgene expression are determined by systemic administration of the peptide-modified AAVpo1 vector in animal models such as mouse models that are well known in the art and disclosed in the examples of the present application. AAVpo1 vector comprising an unmodified capsid and best AAV vector serotypes (AAV2, AAV8, AAV9, AAVrh10, and/or others) commonly used for muscle transduction are advantageously used for comparison. Vector transduction may be determined by measuring vector genome copy number per diploid genome by standard assays that are well known in the art such as real-time PCR assay disclosed in the examples of the present application. Transgene expression is measured at the mRNA or protein levels by standard assays that are well known in the art such as quantitative RT-PCR assay and quantitative western blot analysis as disclosed in the examples of the present application.

In some embodiments, the peptide-modified AAVpo1 vector for the use according to the invention is detargeted from the liver and at least another non-target organ such as the spleen.

In some embodiments, the peptide-modified AAVpo1 vector for the use according to the invention advantageously produces high transgene expression levels in different muscle groups, preferably including major muscle groups, following systemic administration, in particular intravenous administration. The major skeletal muscle groups forming the upper human body are the abdominal, pectoral, deltoid, trapezius, latissimus dorsi, erector spinae, biceps, triceps and diaphragm. The major skeletal muscle groups of the lower human body are the quadriceps, hamstrings, gastrocnemius, soleus, and gluteus. Muscles of the anterior part of the lower leg are the tibialis anterior, extensor digitorium longus, extensor hallucis longus, fibularis longus, fibularis brevis and fibularis tertius. The capacity of the peptide-modified AAVpo1 vector to produce high transgene expression levels in different muscle groups after systemic administration is illustrated in the examples of the present application (FIG. 5) showing high transgene expression levels in the Tibialis (TA), Extensor Digitorum Longus (EDL), Quadriceps (Qua), Gastrocnemius (Ga), Soleus (Sol), Triceps, Biceps and Diaphragm of mice injected intravenously with the peptide-modified AAVpo1 vector.

In some embodiments, the peptide-modified AAVpo1 vector for use according to the invention is characterized by the combination of liver detargeting and transgene expression levels in different muscle groups and in the brain and spinal cord that are at least equivalent if not superior to that of AAV9 vector, after systemic administration, in particular intravenous administration.

AAVpo1 (GenBank accession number FJ688147 as accessed on 24 Jul. 2016) comprises the Cap gene from position 780 to 2930 of the partial viral genome sequence (2977 bp): VP1 CDS is from positions 780 to 2930; VP2 CDS is from positions 1188 to 2930 and VP3 CDS is from positions 1329 to 2930. The AAVpo1 capsid protein (VP1) has the sequence GenBank accession number ACN42940.1 as accessed on 24 Jul. 2016 or SEQ ID NO: 1. Hybrid vectors include for example vectors comprising AAVpo1 capsid and AAV2 rep proteins and/or AAV2 ITRs. AAVpo1 serotype includes the natural AAVpo1 serotype as listed above as well as any artificial variant or hybrid derived from said serotype. The invention encompasses the use of a peptide-modified AAVpo1 vector derived from an AAV capsid sequence having at least at least 95%, 96%, 97%, 98% or 99% identity with AAVpo1 capsid sequence as listed above.

In some embodiments, the peptide-modified AAVpo1 capsid protein is derived from an AAV capsid sequence having at least 95%, 96%, 97%, 98% or 99% identity with the sequence. SEQ ID NO: 1.

The term “identity” refers to the sequence similarity between two polypeptide molecules or between two nucleic acid molecules. When a position in both compared sequences is occupied by the same base or same amino acid residue, then the respective molecules are identical at that position. The percentage of identity between two sequences corresponds to the number of matching positions shared by the two sequences divided by the number of positions compared and multiplied by 100. Generally, a comparison is made when two sequences are aligned to give maximum identity. The identity may be calculated by alignment using, for example, the GCG (Genetics Computer Group, Program Manual for the GCG Package, Version 7, Madison, Wisconsin) pileup program, or any of sequence comparison algorithms such as BLAST, FASTA or CLUSTALW.

The peptide is preferably of up to 30 amino acids. In some preferred embodiments, the peptide is of up to 25, 20 or 15 amino acids (i.e., 25, 24, 23, 22, 21, 20, 19, 18, 17, 16, or 15 amino acids).

In some embodiments, the peptide, preferably of up to 30 amino acids, comprises or consists of the sequence MPLGAAG (SEQ ID NO: 2) or a variant comprising only one or two amino acid mutations (insertion, deletion, substitution) in said sequence, preferably one or two amino acid substitutions in said sequence. In some preferred embodiments, the peptide comprises or consists of the sequence GMPLGAAGA (SEQ ID NO: 3), or a variant comprising up to four (1, 2, 3 or 4) amino acid mutations (insertion, deletion, substitution) in said sequence, preferably only one or two amino acid deletions or substitutions in said sequence ; said deletions being advantageously at the N- and/or C-terminal ends. In some preferred embodiments, the sequence SEQ ID NO: 2 or 3 or variant thereof as defined above is flanked by up to five (1, 2, 3, 4, 5) or more amino acids at its N-and/or C-terminal ends, such as GQR and QAA, respectively at its N-and C-terminal ends. Alternatively, the flanking sequences may comprise or consist of alanine (A) residues. In some more preferred embodiments, the peptide comprises or consists of the sequence GQRGMPLGAAGAQAA (SEQ ID NO: 4).

The peptide-modified AAVpo1 capsid protein comprises at least one copy of the peptide inserted into AAVpo1 capsid protein. Depending on the position of the insertion, the peptide may be inserted into VP1, VP1 and VP2 or VP1, VP2 and VP3. The peptide-modified AAVpo1 capsid protein may comprise up to 5 copies of the peptide, preferably 1 copy of said peptide.

The peptide-modified AAVpo1 capsid protein according to invention comprises the one or more peptide(s) inserted into a site exposed on the AAV capsid surface. Sites on the AAV capsid which are exposed on the capsid surface and tolerate peptide insertions, i.e. do not affect assembly and packaging of the virus capsid, are well-known in the art and include for example the AAV capsid surface loops or antigenic loops (Girod et al., Nat. Med., 1999, 5, 1052-1056; Grifman et al., Molecular Therapy, 2001, 3, 964-975); other sites are disclosed in Rabinowitz et al., Virology, 1999, 265, 274-285; Wu et al., J. Virol., 2000, 74, 8635-8647.

In particular, the at least one peptide is inserted at any of positions N567, S568, N569, T570 of the capsid protein according to the numbering in SEQ ID NO: 1, preferably between positions N567 and S568 or between positions N569 and T570 of the capsid protein. The insertion of the peptide may or may not cause the deletion of some residues preceding and/or following the peptide insertion site, preferably one to three (1, 2, 3) of said residues. In some embodiments the peptide is inserted between positions N567 and S568 and replaces all the residues from positions 565-567 and 568-570. In some other embodiments the peptide is inserted between positions N569 and T570 and replaces all the residues from positions from positions 567-569 and 570-572. The positions are indicated by reference to AAVpo1 capsid protein of SEQ ID NO: 1; one skilled in the art will be able to find easily the corresponding positions in another AAVpo1 capsid protein sequence after alignment with SEQ ID NO: 1.

In some preferred embodiments, said peptide-modified AAVpo1 capsid protein comprises a sequence selected from the group consisting of the sequence SEQ ID NO: 5 and the sequences having at least 95%, 96%, 97%, 98% or 99% identity with SEQ ID NO: 5 which comprise said peptide according to the present disclosure, and the fragment thereof corresponding to VP2 or VP3 capsid protein. VP2 corresponds to the amino acid sequence from K136 to the end of SEQ ID NO: 5. VP3 corresponds to the amino acid sequence from M184 to the end of SEQ ID NO: 5. In some preferred embodiments, said peptide-modified AAVpo1 capsid protein comprises the sequence SEQ ID NO: 5, or a fragment thereof corresponding to VP2 or VP3 capsid protein.

The invention encompasses also AAVpo1 VP1 and VP2 chimeric capsid proteins derived from the peptide-modified AAVpo1 VP3 capsid protein according to the disclosure, wherein the VP1-specific N-terminal region and/or VP2-specific N-terminal region are from another natural or artificial AAV serotype, preferably another AAVpo serotype chosen from the known AAVpo serotypes, in particular AAVpo2.1 serotype. The invention further encompasses mosaic peptide-modified AAVpo1 vectors, wherein the vector particle further comprises another AAV capsid protein from another natural or artificial AAV serotype preferably another AAVpo serotype chosen from known AAVpo serotypes, in particular AAVpo2.1 serotype according to the present disclosure.

The genome of the peptide-modified AAVpo1 vector may either be a single-stranded or self-complementary double-stranded genome (McCarty et al, Gene Therapy, 2003, Dec.,10(26), 2112-2118). Self-complementary vectors are generated by deleting the terminal resolution site (trs) from one of the AAV terminal repeats. These modified vectors, whose replicating genome is half the length of the wild-type AAV genome have the tendency to package DNA dimers. The AAV genome is flanked by ITRs. In particular embodiments, the AAV vector is a pseudotyped vector, i.e. its genome and capsid are derived from AAVs of different serotypes. In some preferred embodiments, the genome of the pseudotyped vector is derived from AAV2.

The peptide-modified AAVpo1 vector for use according to the present disclosure is produced by standard methods for producing AAV vectors that are well-known in the art (Review in Aponte-Ubillus et al., Applied Microbiology and Biotechnology, 2018, 102: 1045-1054). Briefly, following co-transfection with expression plasmid(s) for AAV Rep and Capsid proteins and plasmid containing recombinant AAV vector genome comprising the gene of interest inserted in an expression cassette, flanked by AAV ITRs, in the presence of sufficient helper function to permit packaging of the rAAV vector genome into AAV capsid particle, the cells are incubated for a time sufficient to allow the production of AAV vector particles, the cells are then harvested, lysed, and AAV vector particles are purified by standard purification methods such as affinity chromatography or Iodixanol or Cesium Chloride density gradient ultracentrifugation.

The peptide-modified AAVpo1 vector particle usually packages a gene of interest for therapy. By “gene of interest for therapy”, “gene of therapeutic interest”, “gene of interest” or “heterologous gene of interest”, it is meant a therapeutic gene or a gene encoding a therapeutic protein, peptide or RNA. The therapeutic gene may be used in combination with a genome-editing enzyme.

The gene of interest is any nucleic acid sequence capable of modifying a target gene or target cellular pathway, in cells of target organs (i.e. nervous system, such as CNS; or muscles and/or nervous system such as muscles and CNS). Depending on the type of disease, the target organs may comprise essentially the nervous system such as the CNS or may further comprise muscles. In some particular embodiments, the target organ comprises at least the nervous system, such as the CNS. In some preferred embodiments, the target organ comprises the nervous system and muscles, such as the CNS and muscles. For example, the gene may modify the expression, sequence or regulation of the target gene or cellular pathway. In some embodiments, the gene of interest is a functional version of a gene or a fragment thereof. The functional version of said gene includes the wild-type gene, a variant gene such as variants belonging to the same family and others, or a truncated version, which preserves the functionality of the encoded protein at least partially. A functional version of a gene is useful for replacement or additive gene therapy to replace a gene, which is deficient or non-functional in a patient. In other embodiments, the gene of interest is a gene which inactivates a dominant allele causing an autosomal dominant genetic disease. A fragment of a gene is useful as recombination template for use in combination with a genome editing enzyme.

Alternatively, the gene of interest may encode a protein of interest for a particular application, (for example an antibody or antibody fragment, a genome-editing enzyme) or a RNA. In some embodiments, the protein is a therapeutic protein including a therapeutic antibody or antibody fragment, or a genome-editing enzyme. In some embodiments, the RNA is a therapeutic RNA.

In some embodiments, the sequence of the gene of interest is optimized for expression in the treated individual, preferably a human individual. Sequence optimization may include a number of changes in a nucleic acid sequence, including codon optimization, increase of GC content, decrease of the number of CpG islands, decrease of the number of alternative open reading frames (ARFs) and/or decrease of the number of splice donor and splice acceptor sites.

The gene of interest is a functional gene able to produce the encoded protein, peptide or RNA in the target cells of the disease, in particular muscle cells and cells of the nervous system (CNS and/or PNS), such as muscle cells and cells of the CNS. Depending on the type of disease, the target cells may comprise essentially nervous system cells, such as CNS cells or may further comprise muscle cells. In some particular embodiments, the target cells of the disease comprise at least nervous system cells, such as CNS cells. In some preferred embodiments, the target cells of the disease comprise nervous system cells and muscle cells, such as CNS cells and muscle cells. In some embodiments, the gene of interest is a human gene. The peptide-modified AAVpo1 vector comprises the gene of interest in a form expressible in cells of target organs (i.e. nervous system, such as CNS; or muscles and/or nervous system, such as muscles and/or CNS). In some particular embodiments, the gene of interest is in a form expressible at least in nervous system cells, such as CNS cells. In some preferred embodiments, the gene of interest is in a form expressible in nervous system cells and muscle cells, such as CNS cells and muscle cells. In particular, the gene of interest is operably linked to appropriate regulatory sequences for expression of a transgene in the individual’s target cells, tissue(s) or organ(s). Such sequences which are well-known in the art include in particular a promoter, and further regulatory sequences capable of further controlling the expression of a transgene, such as without limitation, enhancer, terminator, intron, silencer, in particular tissue-specific silencer, and microRNA. The gene of interest is operably linked to a ubiquitous, tissue-specific or inducible promoter which is functional in cells of target organs (i.e. muscles and/or nervous system, such as muscles and/or CNS). In some particular embodiments, the target organ comprises at least the nervous system, such as the CNS. In some preferred embodiments, the target organ comprises the nervous system and muscles, such as the CNS and muscles. In some particular embodiments, the gene of interest is operably linked to a ubiquitous, tissue-specific or inducible promoter which is functional in nervous system cells, such as neurons and/or glial cells; or in nervous system cells, such as such as neurons and/or glial cells, and in muscular cells. In some particular embodiments, the gene of interest is operably linked to at least two promoters, wherein at least one of them is neurons and/or glial cells specific- or inducible promoter which is functional in neurons and/or glial cells. In some particular embodiments, the gene of interest is operably linked to at least two promoters wherein one of them is neurons and/or glial cells specific- or inducible promoter which is functional in neurons and/or glial cells and the other is muscle-specific or inducible promoter which is functional in muscular cells.

The gene of interest may be inserted in an expression cassette further comprising additional regulatory sequences as disclosed above. Examples of ubiquitous promoters include the CAG promoter, phosphoglycerate kinase 1 (PGK) promoter, the cytomegalovirus enhancer/promoter (CMV), the SV40 early promoter, the retroviral Rous sarcoma virus (RSV) LTR promoter, the dihydrofolate reductase promoter, the β-actin promoter, and the EF1 promoter.

Muscle-specific promoters include without limitation, the desmin (Des) promoter, muscle creatine kinase (MCK) promoter, CK6 promoter, alpha-myosin heavy chain (alpha-MHC) promoter, myosin light chain 2 (MLC-2) promoter, cardiac troponin C (cTnC) promoter, synthetic muscle-specific SpC5-12 promoter, the human skeletal actin (HSA) promoter.

Promoters for nervous system, such as CNS expression include promoters driving ubiquitous expression and promoters driving expression into neurons. Representative promoters driving ubiquitous expression, without limitation: CAG promoter (includes the cytomegalovirus enhancer/chicken beta actin promoter, the first exon and the first intron of the chicken beta-actin gene and the splice acceptor of the rabbit beta-globin gene); PGK (phosphoglycerate kinase 1) promoter; β-actin promoter; EF1a promoter; CMV promoter. Representative promoters driving expression into neurons include, without limitation, the promoter of the Calcitonin Gene-Related Peptide (CGRP), a known motor neuron-derived factor. Other neuron-selective promoters include the promoters of Choline Acetyl Transferase (ChAT), Neuron Specific Enolase (NSE), Synapsin, Hb9 and ubiquitous promoters including Neuron-Restrictive Silencer Elements (NRSE). Representative promoters driving selective expression in glial cells include the promoter of the Glial Fibrillary Acidic Protein gene (GFAP).

For expression in muscle cells (skeletal and cardiac muscle cells), the gene of interest is advantageously under the control of a desmin promoter, in particular human desmin promoter (Raguz et al., Dev. Biol., 1998, 201, 26-42; Paulin D & Li Z, Exp. Cell. Res., 2004, Nov 15;301(1):1-7). For expression in skeletal muscle cells, the gene of interest is advantageously under the control of a desmin promoter, in particular human desmin promoter, and further comprises a miR208a target sequence that represses expression in cardiac muscle cells (i.e. in the heart; Roudault et al., Circulation, 2013, 128, 1094-104. doi: 10.1161/CIRCULATIONAHA.113.001340).

The RNA is advantageously complementary to a target DNA or RNA sequence or binds to a target protein. For example, the RNA is an interfering RNA such as a shRNA, a microRNA, a guide RNA (gRNA) for use in combination with a Cas enzyme or similar enzyme for genome editing, an antisense RNA capable of exon skipping such as a modified small nuclear RNA (snRNA) or a long non-coding RNA. The interfering RNA or microRNA may be used to regulate the expression of a target gene involved in muscle or nervous system disease, such as muscle or CNS disease. In some embodiments, the disease is a nervous system disease, such as CNS disease. According to this embodiment, the target gene is in nervous system cells, such as CNS and/or PNS cells, including in particular neurons and/or glial cells. In some other embodiments, the disease is a disease of the nervous system and muscles, such as a disease of the CNS and muscles. According to this other embodiment, the target gene is at least in nervous system cells such as CNS and/or PNS cells, including in particular neurons and/or glial cells; the target gene may be essentially in nervous system cells such as CNS and/or PNS cells, including in particular neurons and/or glial cells; or may be in nervous system cells and muscle cells, such as CNS and/or PNS cells, including in particular neurons and/or glial cells, and muscle cells. The guide RNA in complex with a Cas enzyme or similar enzyme for genome editing may be used to modify the sequence of a target gene, in particular to correct the sequence of a mutated/deficient gene or to modify the expression of a target gene involved in a disease, in particular a muscle or nervous system disorder, such as muscle or central nervous system (CNS) disorder. The antisense RNA capable of exon skipping is used in particular to correct a reading frame and restore expression of a deficient gene having a disrupted reading frame. In some embodiments, the RNA is a therapeutic RNA.

The genome-editing enzyme according to the invention is any enzyme or enzyme complex capable of modifying a target gene or target cellular pathway, in particular in muscle cells and/or cells of the nervous system, such as muscle cells and/or cells of the CNS. In some embodiments, the target gene is at least in nervous system cells, such as CNS and/or PNS cells, including in particular neurons and/or glial cells; the target gene may be essentially in nervous system such as CNS and/or PNS cells, including in particular neurons and/or glial cells; or may be in nervous system cells and muscle cells, such as CNS and/or PNS cells, including in particular neurons and/or glial cells, and muscle cells. In some particular embodiments, the target gene is in nervous system cells, such as CNS and/or PNS cells, including in particular neurons and/or glial cells. In some other particular embodiments, the target gene is in nervous system cells and muscle cells, such as CNS and/or PNS cells, including in particular neurons and/or glial cells, and muscle cells. For example, the genome-editing enzyme may modify the expression, sequence or regulation of the target gene or cellular pathway.The genome-editing enzyme is advantageously an engineered nuclease, such as with no limitations, a meganuclease, zinc finger nuclease (ZFN), transcription activator-like effector-based nuclease (TALENs), Cas enzyme from clustered regularly interspaced palindromic repeats (CRISPR)-Cas system and similar enzymes. The genome-editing enzyme, in particular an engineered nuclease such as Cas enzyme and similar enzymes, may be a functional nuclease which generates a double-strand break (DSB) or single-stranded DNA break (nickase such as Cas9(D10A) in the target genomic locus and is used for site-specific genome editing applications, including with no limitations: gene correction, gene replacement, gene knock-in, gene knock-out, mutagenesis, chromosome translocation, chromosome deletion, and the like. For site-specific genome editing applications, the genome-editing enzyme, in particular an engineered nuclease such as Cas enzyme and similar enzymes may be used in combination with a homologous recombination (HR) matrix or template (also named DNA donor template) which modifies the target genomic locus by double-strand break (DSB)-induced homologous recombination. In particular, the HR template may introduce a transgene of interest into the target genomic locus or repair a mutation in the target genomic locus, preferably in an abnormal or deficient gene causing a muscle or nervous system, such as muscle or central nervous system (CNS) disorder. In some embodiments, the disease is a nervous system disease, such as CNS disease. In some other embodiments, the disease is a disease of the nervous system and muscles, such as in particular a disease of the CNS and muscles. Alternatively, the genome-editing enzyme, such as Cas enzyme and similar enzymes may be engineered to become nuclease-deficient and used as DNA-binding protein for various genome engineering applications such as with no limitation: transcriptional activation, transcriptional repression, epigenome modification, genome imaging, DNA or RNA pull-down and the like.

In some embodiment, the peptide-modified AAVpo1 vector particle packaging a gene of interest for therapy targets skeletal muscle cells and/or neurons.

Examples of preferred vectors for use according to the invention is a AAVpo1 vector comprising a peptide-modified capsid protein comprising the sequence SEQ ID NO: 5 or a sequence having at least 95%, 96% , 97%, 98% or 99% identity with said sequence which comprises the peptide of any one of SEQ ID NO : 2 to 4, said vector further packaging a gene of interest for therapy operably linked to a desmin promoter, preferably human desmin promoter and eventually further operably linker to a miR208a target sequence. This first vector is useful for expressing the gene of interest in muscles. (skeletal and cardiac; expression cassette without miR208a target sequence) or only in skeletal muscles (expression cassette with miR208a target sequence) but not in the liver after systemic, in particular intravascular administration.

Another example of preferred vectors for use according to the invention is a AAVpo1 vector comprising a peptide-modified capsid protein comprising the sequence SEQ ID NO: 5 or a sequence having at least 95%, 96% , 97%, 98% or 99% identity with said sequence which comprises the peptide of any one of SEQ ID NO : 2 to 4, said vector further packaging a gene of interest for therapy operably linked to a CAG promoter, and preferably further comprising human beta globin polyadenylation signal. This second vector is useful for expressing the gene of interest in muscles including heart and in the nervous system, such as in muscles including heart and in the CNS but not in the liver after systemic, in particular intravascular administration.

Another example of preferred vectors for use according to the invention is a AAVpo1 vector comprising a peptide-modified capsid protein comprising the sequence SEQ ID NO: 5 or a sequence having at least 95%, 96%, 97%, 98% or 99% identity with said sequence which comprises the peptide of any one of SEQ ID NO : 2 to 4, said vector further packaging a gene of interest for therapy operably linked to a promoter functional in neurons and/or glial cells. The promoter may be a ubiquitous promoter such as CAG or others, tissue-specific promoter, or inducible promoter, which is functional in neurons and/or glial cells. In some particular embodiments, the promoter is neurons and/or glial cells specific- or inducible promoter which is functional in neurons and/or glial cells. This third vector is useful for expressing the gene of interest in the nervous system, but not in the liver after systemic, in particular intravascular administration.

Another example of preferred vectors for use according to the invention is a AAVpo1 vector comprising a peptide-modified capsid protein comprising the sequence SEQ ID NO: 5 or a sequence having at least 95%, 96% , 97%, 98% or 99% identity with said sequence which comprises the peptide of any one of SEQ ID NO : 2 to 4, said vector further packaging a gene of interest for therapy operably linked to a promoter or a combination of promoters that is functional in muscle cells and in neurons and/or glial cells. This fourth vector is useful for expressing the gene of interest in muscles including heart and in the nervous system, such as in muscles including heart and in the CNS but not in the liver after systemic, in particular intravascular administration. The promoter may be a ubiquitous promoter such as CAG or others, tissue-specific promoter or inducible promoter, or a combination of said promoters, including a first promoter functional in muscle cells and a second promoter functional in neurons and/or glial cells. In some particular embodiments, the gene of interest is operably linked to at least two promoters wherein one of them is neurons and/or glial cells specific- or inducible promoter which is functional in neurons and/or glial cells and the other is muscle-specific or inducible promoter which is functional in muscular cells.

### Gene Therapy of Muscle and Nervous System Disorders Such as Muscle and CNS Disorders

The peptide-modified AAVpo1 vector according to the present disclosure is used in the gene therapy of muscle and/or nervous system diseases or disorders, such as muscle and/or CNS diseases or disorders. In some embodiments, the peptide-modified AAVpo1 vector according to the present disclosure is used in the gene therapy of diseases affecting at least the nervous system, such as the CNS, wherein the disease may affect essentially the nervous system, such as the CNS or may affect the nervous system and muscles, such as the CNS and muscles. For example, the disease may primarily affect the nervous system and the primary injury of the nervous system may result in a secondary injury of muscles. In some particular embodiments, the peptide-modified AAVpo1 vector according to the present disclosure is used in the gene therapy of nervous system diseases, in particular CNS diseases. In some other particular embodiments, the peptide-modified AAVpo1 vector according to the present disclosure is used in the gene therapy of diseases of the nervous system and muscles, such as diseases of the CNS and muscles, in particular neuromuscular diseases affecting at least the nervous system (CNS and/or PNS).

The peptide-modified AAVpo1 vector according to the present disclosure is preferably used in the form of a pharmaceutical composition comprising a therapeutically effective amount of peptide-modified AAVpo1 vector particles, preferably peptide-modified AAVpo1 vector particles packaging a therapeutic gene of interest according to the present disclosure.

Gene therapy can be performed by gene transfer, gene editing, exon skipping, RNA-interference, trans-splicing or any other genetic modification of any coding or regulatory sequences in the cell, including those included in the nucleus, mitochondria or as commensal nucleic acid such as with no limitation viral sequences contained in cells.

The two main types of gene therapy are the following:
a therapy aiming to provide a functional replacement gene for a deficient/abnormal gene: this is replacement or additive gene therapy;
a therapy aiming at gene or genome editing: in such a case, the purpose is to provide to a cell the necessary tools to correct the sequence or modify the expression or regulation of a deficient/abnormal gene so that a functional gene is expressed or an abnormal gene is suppressed (inactivated): this is gene editing therapy.

In additive gene therapy, the gene of interest may be a functional version of a gene, which is deficient or mutated in a patient, as is the case for example in a genetic disease. In such a case, the gene of interest will restore the expression of a functional gene.

Gene or genome editing uses one or more gene(s) of interest, such as:
(i) a gene encoding a therapeutic RNA as defined above such as an interfering RNA like a shRNA or a microRNA, a guide RNA (gRNA) for use in combination with a Cas enzyme or similar enzyme, or an antisense RNA capable of exon skipping such as a modified small nuclear RNA (snRNA); and
(ii) a gene encoding a genome-editing enzyme as defined above such as an engineered nuclease like a meganuclease, zinc finger nuclease (ZFN), transcription activator-like effector-based nuclease (TALENs), Cas enzyme or similar enzymes; or a combination of such genes, and maybe also a fragment of a functional version of a gene for use as recombination template, as defined above.

Gene therapy is used for treating various inherited (genetic) or acquired diseases or disorders affecting the structure or function of muscle(s) and/or the nervous system, such as muscle(s) and/or the CNS, including skeletal or cardiac muscle(s), the brain or spinal cord. The diseases may be caused by trauma, infection, degeneration, structural or metabolic defects, tumors, autoimmune disorders, stroke or others. In some embodiments, gene therapy is used for treating inherited (genetic) or acquired diseases or disorders affecting the structure or function of at least the nervous system (PNS and/or CNS), in particular the CNS, including the brain and/or spinal cord. The disease may affect essentially the nervous system (PNS and/or CNS), in particular the CNS, including the brain and/or spinal cord or may further affect muscle(s) including skeletal and/or cardiac muscle(s). In some particular embodiments, the disease is a nervous system disease, in particular a CNS disease and/or a PNS disease; the CNS disease may affect the brain and/or spinal cord. In some other particular embodiments, the disease is a disease of the nervous system (PNS and/or CNS) and muscles, such as a disease of the CNS and muscles; the disease affects the nervous system such as the brain and/or spinal cord and further affects muscle(s) such as skeletal and/or cardiac muscle(s). As used herein, diseases of the nervous system and muscles include diseases with a secondary muscle involvement or injury, in particular as a consequence of a primary involvement or injury of the nervous system in particular the CNS. Therefore, diseases of the nervous system and muscles as disclosed herein are different from muscle diseases which are diseases characterized by a primary muscle injury or involvement.

In some embodiments, gene therapy is used for treating nervous system diseases, in particular CNS diseases, in particular genetic neurological disorders. CNS diseases include for example Alzheimer, Parkinson, Frontotemporal dementia and others.

Examples of mutated genes in genetic neurological disorders, that can be targeted by gene therapy using the pharmaceutical composition of the invention are listed in the following table:

Genetic neurological disorders

| | |
|---|---|
| Disease | Gene |
| Friedreich ataxia | FXN |
| Ataxia-telangectasia | ATM |
| Niemann pick disease | SMPD1, NPC1,2 |
| Spinocerebellar ataxia | ATXN1-3, PLEKHG4, SPTBN2, CACNA1A, ATXN 7 |
| Marinesco-Sjoren | SIL1 |
| Juvenile Parkinsonism | SNCA, PARK2, PINK1, GCH1, TH |
| Dystonia | TOR1A, GCH1 |
| Huntington’s disease | HTT |
| Wilson disease | ATP7B |
| Pantothenate kinase-associated neurodegeneration | PANK2 |
| Lysosomal Storage Disorders (LSDs) | GBA, SMPD1, NPC1, NPC2 |
| Fragile X syndrome | FMR1 |
| Rett Syndrome | MECP2 |
| Adrenoleukodystrophy | ABCD1 |
| Epilepsy | BFNS, KCNQ2, KCNQ3, BFNIS, SCN2A, BFIS, PRRT2, CHRNA4, CHRNB2, CHRNA2, KCNT1, SCNA1, TBC1D24, SCN8A, CDKL5, ARX, STXBP1, SCN1B, GABRG2 |

Other examples of mutated genes in genetic neurological disorders, that can be targeted by gene therapy using the pharmaceutical composition of the invention are the genes responsible for Spinal muscular atrophies (SMAs) & Motor Neuron diseases; Hereditary motor and sensory neuropathies; Hereditary paraplegia and Hereditary ataxia; listed in the tables below. In some particular embodiments, said neurological disease is selected from the group consisting of: Spinal muscular atrophy (SMN1, ASAH1 genes); Amyotrophic lateral sclerosis (SOD1, ALS2, SETX, FUS, ANG, TARDBP, FIG. 4, OPTN and others); Hereditary paraplegia (SPAST (SPG4), SPG7, and other SPG genes such as SPG11, SPG20 and SPG21; in particular SPAST (SPG4) and SPG7) and Charcot-Marie-Tooth, Type 4B1 (MTMR2). In some preferred embodiments, said gene is selected from the group consisting of: SMN1, ASAH1, DNM2, MTMR2 and SPAST genes. In some other preferred embodiments, said gene is selected from the group consisting of: SOD1, ALS2, SETX, FUS, ANG, TARDBP, FIG4 and OPTN.

In some embodiments, gene therapy is used for treating neuromuscular diseases, in particular genetic neuromuscular disorder in humans. Examples of mutated genes in genetic neuromuscular disorders, including genetic muscular disorders that can be targeted by gene therapy using the pharmaceutical composition of the invention are listed in the following tables:

Muscular dystrophies

| | |
|---|---|
| Gene | Protein |
| DMD | Dystrophin |
| EMD | Emerin |
| FHL1 | Four and a half LIM domain 1 |
| LMNA | Lamin A/C |
| SYNE1 | Spectrin repeat containing, nuclear envelope 1 (nesprin 1) |
| SYNE2 | Spectrin repeat containing, nuclear envelope 2 (nesprin 2) |
| TMEM43 | Transmembrane protein 43 |
| TOR1AIP1 | Torsin A interacting protein 1 |
| DUX4 | Double homeobox 4 |
| SMCHD1 | Structural maintenance of chromosomes flexible hinge domain containing 1 |
| PTRF | Polymerase I and transcript release factor |
| MYOT | Myotilin |
| CAV3 | Caveolin 3 |
| DNAJB6 | HSP-40 homologue, subfamily B, number 6 |
| DES | Desmin |
| TNPO3 | Transportin 3 |
| HNRNPDL | Heterogeneous nuclear ribonucleoprotein D-like |
| CAPN3 | Calpain 3 |
| DYSF | Dysferlin |
| SGCG | Gamma sarcoglycan |
| SGCA | Alpha sarcoglycan |
| SGCB | Beta sarcoglycan |
| SGCD | Delta-sarcoglycan |
| TCAP | Telethonin |
| TRIM32 | Tripartite motif-containing 32 |
| FKRP | Fukutin-related protein |
| TTN | Titin |
| POMT1 | Protein-O-mannosyltransferase 1 |
| ANO5 | Anoctamin 5 |
| FKTN | Fukutin |
| POMT2 | Protein-O-mannosyltransferase 2 |
| POMGNT1 | O-linked mannose beta1,2-N-acetylglucosaminyltransferase |
| PLEC | Plectin |
| TRAPPC11 | trafficking protein particle complex 11 |
| GMPPB | GDP-mannose pyrophosphorylase B |
| DAG1 | Dystroglycan1 |
| DPM3 | Dolichyl-phosphate mannosyltransferase polypeptide 3 |
| ISPD | Isoprenoid synthase domain containing |
| VCP | Valosin-containing protein |
| LIMS2 | LIM and senescent cell antigen-like domains 2 |
| GAA | Glucosidase alpha, acid |

Congenital muscular dystrophies

| | |
|---|---|
| Gene | Protein |
| LAMA2 | Laminin alpha 2 chain of merosin |
| COL6A1 | Alpha 1 type VI collagen |
| COL6A2 | Alpha 2 type VI collagen |
| COL6A3 | Alpha 3 type VI collagen |
| SEPN1 | Selenoprotein N1 |
| FHL1 | Four and a half LIM domain 1 |
| ITGA7 | Integrin alpha 7 precursor |
| DNM2 | Dynamin 2 |
| TCAP | Telethonin |
| LMNA | Lamin A/C |
| FKTN | Fukutin |
| POMT1 | Protein-O-mannosyltransferase 1 |
| POMT2 | Protein-O-mannosyltransferase 2 |
| FKRP | Fukutin-related protein |
| POMGNT1 | O-linked mannose beta1,2-N-acetylglucosaminyltransferase |
| ISPD | Isoprenoid synthase domain containing |
| POMGNT2 | protein O-linked mannose N-acetylglucosaminyltransferase 2 |
| B3GNT1 | UDP-GlcNAc:betaGal beta-1,3-N-acetylglucosaminyl-transferase 1 |
| GMPPB | GDP-mannose pyrophosphorylase B |
| LARGE | Like-glycosyltransferase |
| DPM1 | Dolichyl-phosphate mannosyltransferase 1, catalytic subunit |
| DPM2 | Dolichyl-phosphate mannosyltransferase polypeptide 2, regulatory subunit |
| ALG13 | UDP-N-acetylglucosami-nyltransferase |
| B3GALNT2 | Beta-1,3-N-acetylgalacto-saminyltransferase 2 |
| TMEM5 | Transmembrane protein 5 |
| POMK | Protein-O-mannose kinase |
| CHKB | Choline kinase beta |
| ACTA1 | Alpha actin, skeletal muscle |
| TRAPPC11 | trafficking protein particle complex 11 |

Congenital myopathies

| | |
|---|---|
| Gene | Protein |
| TPM3 | Tropomyosin 3 |
| NEB | Nebulin |
| ACTA1 | Alpha actin, skeletal muscle |
| TPM2 | Tropomyosin 2 (beta) |
| TNNT1 | Slow troponin T |
| KBTBD13 | Kelch repeat and BTB (POZ) domain containing 13 |
| CFL2 | Cofilin 2 (muscle) |
| KLHL40 | Kelch-like family member 40 |
| KLHL41 | Kelch-like family member 41 |
| LMOD3 | Leiomodin 3 (fetal) |
| SEPN1 | Selenoprotein N1 |
| RYR1 | Ryanodine receptor 1 (skeletal) |
| MYH7 | Myosin, heavy polypeptide 7, cardiac muscle, beta |
| MTM1 | Myotubularin |
| DNM2 | Dynamin 2 |
| BIN1 | Amphiphysin |
| TTN | Titin |
| SPEG | SPEG complex locus |
| MEGF10 | Multiple EGF-like-domains 10 |
| MYH2 | Myosin, heavy polypeptide 2, skeletal muscle |
| MYBPC3 | Cardiac myosin binding protein-C |
| CNTN1 | Contactin-1 |
| TRIM32 | Tripartite motif-containing 32 |
| PTPLA | Protein tyrosine phosphatase-like (3-Hydroxyacyl-CoA dehydratase |
| CACNA1S | Calcium channel, voltage-dependent, L type, alpha 1S subunit |

Distal myopathies

| | |
|---|---|
| Gene symbol | protein |
| DYSF | Dysferlin |
| TTN | Titin |
| GNE | UDP-N-acetylglucosamine-2-epimerase/N-acetylmannosamine kinase |
| MYH7 | Myosin, heavy polypeptide 7, cardiac muscle, beta |
| MATR3 | Matrin 3 |
| TIA1 | Cytotoxic granuleassociated RNA binding protein |
| MYOT | Myotilin |
| NEB | Nebulin |
| CAV3 | Caveolin 3 |
| LDB3 | LIM domain binding 3 |
| ANO5 | Anoctamin 5 |
| DNM2 | Dynamin 2 |
| KLHL9 | Kelch-like homologue 9 |
| FLNC | Filamin C, gamma (actin-binding protein - 280) |
| VCP | Valosin-containing protein |

Other myopathies

| | |
|---|---|
| Gene symbol | protein |
| ISCU | Iron-sulfur cluster scaffold homolog (E. coli) |
| MSTN | Myostatin |
| FHL1 | Four and a half LIM domain 1 |
| BAG3 | BCL2-associated athanogene 3 |
| ACVR1 | Activin A receptor, type II-like kinase 2 |
| MYOT | Myotilin |
| FLNC | Filamin C, gamma (actin-binding protein - 280) |
| LDB3 | LIM domain binding 3 |
| LAMP2 | Lysosomal-associated membrane protein 2 precursor |
| VCP | Valosin-containing protein |
| CAV3 | Caveolin 3 |
| SEPN1 | Selenoprotein N1 |
| CRYAB | Crystallin, alpha B |
| DES | Desmin |
| VMA21 | VMA21 Vacuolar H+-ATPase Homolog (S. Cerevisiae) |
| PLEC | plectin |
| PABPN1 | Poly(A) binding protein, nuclear 1 |
| TTN | Titin |
| RYR1 | Ryanodine receptor 1 (skeletal) |
| CLN3 | Ceroid-lipofuscinosis, neuronal 3 (=battenin) |
| TRIM54 | |
| TRIM63 | Tripartite motif containing 63, E3 ubiquitin protein ligase |

Myotonic syndromes

| | |
|---|---|
| Gene | protein |
| DMPK | Myotonic dystrophy protein kinase |
| CNBP (ZNF9) | Cellular nucleic acid-binding protein |
| CLCN1 | Chloride channel 1, skeletal muscle (Thomsen disease, autosomal dominant) |
| CAV3 | Caveolin 3 |
| HSPG2 | Perlecan |
| ATP2A1 | ATPase, Ca++ transporting, fast twitch 1 |

Ion Channel muscle diseases

| | |
|---|---|
| Gene | protein |
| CLCN1 | Chloride channel 1, skeletal muscle (Thomsen disease, autosomal dominant) |
| SCN4A | Sodium channel, voltage-gated, type IV, alpha |
| SCN5A | Voltage-gated sodium channel type V alpha |
| CACNA1S | Calcium channel, voltage-dependent, L type, alpha 1S subunit |
| CACNA1A | Calcium channel, voltage-dependent, P/Q type, alpha 1A subunit |
| KCNE3 | Potassium voltage-gated channel, Isk-related family, member 3 |
| KCNA1 | Potassium voltage-gated channel, shaker-related subfamily, member 1 |
| KCNJ18 | Kir2.6 (inwardly rectifying potassium channel 2.6) |
| KCNJ2 | Potassium inwardly-rectifying channel J2 |
| KCNH2 | Voltage-gated potassium channel, subfamily H, member 2 |
| KCNQ1 | Potassium voltage-gated channel, KQT-like subfamily, member 1 |
| KCNE2 | Potassium voltage-gated channel, Isk-related family, member 2 |
| KCNE1 | Potassium voltage-gated channel, Isk-related family, member 1 |

Malignant hyperthermia

| | |
|---|---|
| Gene | protein |
| RYR1 | Ryanodine receptor 1 (skeletal) |
| CACNA1S | Calcium channel, voltage-dependent, L type, alpha 1S subunit |

Metabolic myopathies

| | |
|---|---|
| Gene | protein |
| GAA | Acid alpha-glucosidase preproprotein |
| AGL | Amylo-1,6-glucosidase, 4-alpha-glucanotransferase |
| GBE1 | Glucan (1,4-alpha-), branching enzyme 1 (glycogen branching enzyme, Andersen disease, glycogen storage disease type IV) |
| PYGM | Glycogen phosphorylase |
| PFKM | Phosphofructokinase, muscle |
| PHKA1 | Phosphorylase b kinase, alpha submit |
| PGM1 | Phosphoglucomutase 1 |
| GYG1 | Glycogenin 1 |
| GYS1 | Glycogen synthase 3 glycogen synthase 1 (muscle) glycogen synthase 1 (muscle) |
| PRKAG2 | Protein kinase, AMP-activated, gamma 2 non-catalytic subunit |
| RBCK1 | RanBP-type and C3HC4-type zinc finger containing 1 (heme-oxidized IRP2 ubiquitin ligase 1) |
| PGK1 | Phosphoglycerate kinase 1 |
| PGAM2 | Phosphoglycerate mutase 2 (muscle) |
| LDHA | Lactate dehydrogenase A |
| ENO3 | Enolase 3, beta muscle specific |
| CPT2 | Carnitine palmitoyltransferase II |
| SLC22A5 | Solute carrier family 22 member 5 |
| SLC25A20 | Carnitine-acylcarnitine translocase |
| ETFA | Electron-transfer-flavoprotein, alpha polypeptide |
| ETFB | Electron-transfer-flavoprotein, beta polypeptide |
| ETFDH | Electron-transferring-flavoprotein dehydrogenase |
| ACADVL | Acyl-Coenzyme A dehydrogenase, very long chain |
| ABHD5 | Abhydrolase domain containing 5 |
| PNPLA2 | Adipose triglyceride lipase (desnutrin) |
| LPIN1 | Lipin 1 (phosphatidic acid phosphatase 1) |
| PNPLA8 | Patatin-like phospholipase domain containing 8 |

Hereditary Cardiomyopathies

| | |
|---|---|
| Gene | protein |
| MYH6 | Myosin heavy chain 6 |
| MYH7 | Myosin, heavy polypeptide 7, cardiac muscle, beta |
| TNNT2 | Troponin T2, cardiac |
| TPM1 | Tropomyosin 1 (alpha) |
| MYBPC3 | Cardiac myosin binding protein-C |
| PRKAG2 | Protein kinase, AMP-activated, gamma 2 non-catalytic subunit |
| TNNI3 | Troponin I, cardiac |
| MYL3 | Myosin light chain 3 |
| TTN | Titin |
| MYL2 | Myosin light chain 2 |
| ACTC1 | Actin, alpha, cardiac muscle precursor |
| CSRP3 | Cysteine and glycine-rich protein 3 (cardiac LIM protein) |
| TNNC1 | Slow troponin C |
| VCL | Vinculin |
| MYLK2 | Myosin light chain kinase 2 |
| CAV3 | Caveolin 3 |
| MYOZ2 | Myozenin 2, or calsarcin 1, a Z disk protein |
| JPH2 | Junctophilin-2 |
| PLN | Phospholamban |
| NEXN | Nexilin(F-actin binding protein) |
| ANKRD1 | Ankyrin repeat domain 1 (cardiac muscle) |
| ACTN2 | Actinin alpha2 |
| NDUFAF1 | NADH-ubiquinone oxidoreductase 1 alpha subcomplex |
| TSFM | Ts translation elongation factor, mitochondrial |
| AARS2 | Alanyl-tRNA synthetase 2, mitochondrial |
| MRPL3 | Mitochondrial ribosomal protein L3 |
| COX15 | COX15 homolog, cytochrome c oxidase assembly protein (yeast) |
| MTO1 | Mitochondrial tRNA translation optimization 1 |
| MRPL44 | Mitochondrial ribosomal protein L44 |
| LMNA | Lamin A/C |
| LDB3 | LIM domain binding 3 |
| SCN5A | Voltage-gated sodium channel type V alpha |
| DES | Desmin |
| EYA4 | Eyes absent 4 |
| SGCD | Delta-sarcoglycan |
| TCAP | Telethonin |
| ABCC9 | ATP-binding cassette, sub-family C (member 9) |
| TMPO | Lamina-associated polypeptide 2 |
| PSEN2 | Presenilin 2 |
| CRYAB | Crystallin, alpha B |
| FKTN | Fukutin |
| TAZ | Tafazzin |
| DMD | Dystrophin |
| LAMA4 | Laminin alpha 4 |
| ILK | Integrin-linked kinase |
| MYPN | Myopalladin |
| RBM20 | RNA binding motif protein 20 |
| SYNE1 | Spectrin repeat containing, nuclear envelope 1 (nesprin 1) |
| MURC | Muscle-related coiled-coil protein |
| DOLK | Dolichol kinase |
| GATAD1 | GATA zinc finger domain containing 1 |
| SDHA | succinate dehydrogenase complex, subunit A, flavoprotein (Fp) |
| GAA | Acid alpha-glucosidase preproprotein |
| DTNA | Dystrobrevin, alpha |
| FLNA | Filamin A, alpha (actin binding protein 280) |
| TGFB3 | Transforming growth factor, beta 3 |
| RYR2 | Ryanodine receptor 2 |
| TMEM43 | Transmembrane protein 43 |
| DSP | Desmoplakin |
| PKP2 | Plakophilin 2 |
| DSG2 | Desmoglein 2 |
| DSC2 | Desmocollin 2 |
| JUP | Junction plakoglobin |
| CASQ2 | Calsequestrin 2 (cardiac muscle) |
| KCNQ1 | Potassium voltage-gated channel, KQT-like subfamily, member 1 |
| KCNH2 | Voltage-gated potassium channel, subfamily H, member 2 |
| ANK2 | Ankyrin 2 |
| KCNE1 | Potassium voltage-gated channel, Isk-related family, member 1 |
| KCNE2 | Potassium voltage-gated channel, Isk-related family, member 2 |
| KCNJ2 | Potassium inwardly-rectifying channel J2 |
| CACNA1C | Calcium channel, voltage-dependent, L type, alpha 1C subunit |
| SCN4B | Sodium channel, voltage-gated, type IV, beta subunit |
| AKAP9 | A kinase (PRKA) anchor protein (yotiao) 9 |
| SNTA1 | Syntrophin, alpha 1 |
| KCNJ5 | Potassium inwardly-rectifying channel, subfamily J, member 5 |
| NPPA | Natriuretic peptide precursor A |
| KCNA5 | Potassium voltage-gated channel, shaker-related subfamily, member 5 |
| GJA5 | Connexin 40 |
| SCN1B | Sodium channel, voltage-gated, type I, beta subunit |
| SCN2B | Sodium channel, voltage-gated, type II, beta subunit |
| NUP155 | Nucleoporin 155 kDa |
| GPD1L | Glycerol-3-phosphate dehydrogenase 1-like |
| CACNB2 | Calcium channel, voltage-dependent, beta 2 subunit |
| KCNE3 | Potassium voltage-gated channel, Isk-related family, member 3 |
| SCN3B | Sodium channel, voltage-gated, type III, beta subunit |
| HCN4 | Hyperpolarization activated cyclic nucleotide-gated potassium channel 4 |

Congenital myasthenic syndromes

| | |
|---|---|
| Gene | protein |
| CHRNA1 | Cholinergic receptor, nicotinic, alpha polypeptide 1 |
| CHRNB1 | Cholinergic receptor, nicotinic, beta 1 muscle |
| CHRND | Cholinergic receptor, nicotinic, delta |
| CHRNE | Cholinergic receptor, nicotinic, epsilon |
| RAPSN | Rapsyn |
| CHAT | Choline acetyltransferase isoform |
| COLQ | Acetylcholinesterase collagen-like tail subunit |
| MUSK | muscle, skeletal, receptor tyrosine kinase |
| DOK7 | Docking protein 7 |
| AGRN | Agrin |
| GFPT1 | Glutamine-fructose-6-phosphate transaminase 1 |
| DPAGT1 | Dolichyl-phosphate (UDP-N-acetylglucosamine) N-acetylglucosaminephosphotransferase 1 (GlcNAc-1-P transferase) |
| LAMB2 | Laminin, beta 2 (laminin S) |
| SCN4A | Sodium channel, voltage-gated, type IV, alpha |
| CHRNG | Cholinergic receptor, nicotinic, gamma polypeptide |
| PLEC | plectin |
| ALG2 | Alpha-1,3/1,6-mannosyltransferase |
| ALG14 | UDP-N-acetylglucosaminyltransferase |
| SYT2 | Synaptotagmin II |
| PREPL | Prolyl endopeptidase-like |

Spinal muscular atrophies (SMAs) &Motor Neuron diseases

| | |
|---|---|
| Gene | protein |
| SMN1 | Survival of motor neuron 1, telomeric |
| IGHMBP2 | Immunoglobulin mu binding protein 2 |
| PLEKHG5 | Pleckstrin homology domain containing, family G (with RhoGef domain) member 5 |
| HSPB8 | Heat shock 27 kDa protein 8 |
| HSPB1 | Heat shock 27 kDa protein 1 |
| HSPB3 | Heat shock 27 kDa protein 3 |
| AARS | Alanyl-tRNA synthetase |
| GARS | Glycyl-tRNA synthetase |
| BSCL2 | Seipin |
| REEP1 | Receptor accessory protein 1 |
| SLC5A7 | Solute carrier family 5 (sodium/choline cotransporter), member 7 |
| DCTN1 | Dynactin 1 |
| UBA1 | Ubiquitin-activating enzyme 1 |
| ATP7A | ATPase, Cu++ transporting, alpha polypeptide |
| DNAJB2 | DnaJ (Hsp40) homolog, subfamily B, member 2 |
| TRPV4 | Transient receptor potential cation channel, subfamily V, member 4 |
| DYNC1H1 | Dynein, cytoplasmic 1, heavy chain 1 |
| BICD2 | Bicaudal D homolog 2 (Drosophila) |
| FBX038 | F-box protein 38 |
| ASAH1 | N-acylsphingosine amidohydrolase (acid ceramidase) 1 |
| VAPB | Vesicle-associated membrane protein-associated protein B and C |
| EXOSC8 | Exosome component 8 |
| SOD1 | Superoxide dismutase 1, soluble |
| ALS2 | Alsin |
| SETX | Senataxin |
| FUS | Fusion (involved in t(12;16) in malignant liposarcoma) |
| ANG | Angiogenin |
| TARDBP | TAR DNA binding protein |
| FIG4 | Sac domain-containing inositol phosphatase 3 |
| OPTN | Optineurin |
| ATXN2 | Ataxin 2 |
| VCP | Valosin-containing protein |
| UBQLN2 | Ubiquilin 2 |
| SIGMAR1 | Sigma non-opioid intracellular receptor 1 |
| CHMP2B | Charged multivesicular body protein 2B |
| PFN1 | Profilin 1 |
| MATR3 | Matrin 3 |
| NEFH | Neurofilament, heavy polypeptide |
| PRPH | Peripherin |
| C9orf72 | Chromosome 9 open reading frame 72 |
| CHCHD10 | Coiled-coil-helix-coiled-coil-helix domain containing 10 |
| SQSTM1 | Sequestosome 1 |
| AR | Androgen receptor |
| GLE1 | GLE1 RNA export mediator homolog (yeast) |
| ERBB3 | V-erb-b2 erythroblastic leukemia viral oncogene homolog 3 (avian) |
| PIP5K1C | Phosphatidylinositol-4-phosphate 5-kinase, type I, gamma |
| EXOSC3 | Exosome component 3 |
| VRK1 | Vaccinia related kinase 1 |
| SLC52A3 | Solute carrier family 52, riboflavin transporter, member 3 |
| SLC52A2 | Solute carrier family 52, riboflavin transporter, member 2 |
| HEXB | Hexosaminidase B |

Hereditary motor and sensory neuropathies

| | |
|---|---|
| Gene | Protein |
| PMP22 | Peripheral myelin protein 22 |
| MPZ | Myelin protein zero |
| LITAF | Lipopolysaccharide-induced TNF factor |
| EGR2 | Early growth response 2 protein |
| NEFL | Neurofilament, light polypeptide 68 kDa |
| HOXD10 | Homeobox D10 |
| ARHGEF10 | Rho guanine nucleotide exchange factor 10 |
| FBLN5 | Fibulin 5 (extra-cellular matrix) |
| DNM2 | Dynamin 2 |
| YARS | Tyrosyl-tRNA synthetase |
| INF2 | Inverted formin 2 |
| GNB4 | Guanine nucleotidebinding protein, beta-4 |
| GDAP1 | Ganglioside-induced differentiation-associated protein 1 |
| MTMR2 | Myotubularin-related protein 2 |
| SBF2 | SET binding factor 2 |
| SBF1 | SET binding factor 1 |
| SH3TC2 | KIAA1985 protein |
| NDRG1 | N-myc downstream regulated gene 1 |
| PRX | Periaxin |
| HK1 | Hexokinase 1 |
| FGD4 | Actin-filament binding protein Frabin |
| FIG4 | Sac domain-containing inositol phosphatase 3 |
| SURF1 | surfeit 1 |
| GJB1 | Gap junction protein, beta 1, 32 kDa (connexin 32) |
| AIFM1 | Apoptosis-inducing factor, mitochondrionassociated 1 |
| PRPS1 | Phosphoribosyl pyrophosphate synthetase 1 |
| PDK3 | Pyruvate dehydrogenase kinase, isoenzyme 3 |
| KIF1B | Kinesin family member 1B |
| MFN2 | Mitofusin 2 |
| RAB7A | RAB7, member RAS oncogene family |
| TRPV4 | Transient receptor potential cation channel, subfamily V, member 4 |
| GARS | Glycyl-tRNA synthetase |
| HSPB1 | Heat shock 27 kDa protein 1 |
| HSPB8 | Heat shock 27 kDa protein 8 |
| AARS | Alanyl-tRNA synthetase |
| DYNC1H1 | Dynein, cytoplasmic 1, heavy chain 1 |
| LRSAM1 | leucine rich repeat and sterile alpha motif containing 1 |
| DHTKD1 | dehydrogenase E1 and transketolase domain containing 1 |
| TRIM2 | Tripartite motif containing 2 |
| TFG | TRK-fused gene |
| MARS | methionyl-tRNA synthetase |
| KIF5A | Kinesin family member 5A |
| LMNA | Lamin A/C |
| MED25 | Mediator complex subunit 25 |
| DNAJB2 | DnaJ (Hsp40) homolog, subfamily B, member 2 |
| HINT1 | Histidine triad nucleotide binding protein 1 |
| KARS | Lysyl-tRNA synthetase |
| PLEKHG5 | Pleckstrin homology domain containing, family G (with RhoGef domain) member 5 |
| COX6A1 | Cytochrome c oxidase subunit VIa polypeptide 1 |
| IGHMBP2 | Immunoglobulin mu binding protein 2 |
| SPTLC1 | Serine palmitoyltransferase subunit 1 |
| SPTLC2 | Serine palmitoyltransferase long chain base subunit 2 |
| ATL1 | Atlastin GTPase 1 |
| KIF1A | Kinesin family member 1A |
| WNK1 | WNK lysine deficient protein kinase 1 |
| IKBKAP | Inhibitor of kappa light polypeptide gene enhancer in B-cells, kinase complex-associated protein |
| NGF | Nerve growth factor (beta polypeptide) |
| DNMT1 | DNA (cytosine-5)-methyltransferase 1 |
| SLC12A6 | Potassium chloride cotransporter KCC3 |
| GJB3 | Gap junction protein, beta 3, 31 kDa (=connexin 31) |
| sept-09 | Septin 9 |
| GAN | Gigaxonin |
| CTDP1 | CTD phosphatase subunit 1 |
| VRK1 | Vaccinia related kinase 1 |

Hereditary paraplegia

| | |
|---|---|
| Gene symbol | protein |
| ATL1 | Atlastin |
| SPAST | Spastin |
| NIPA1 | Non-imprinted in Prader-Willi/Angelman syndrome 1 |
| KIAA0196 | Strumpellin |
| KIF5A | Kinesin family member 5A |
| RTN2 | Reticulon 2 |
| HSPD1 | Heat shock 60 kDa protein 1 (chaperonin) |
| BSCL2 | Seipin |
| REEP1 | Receptor accessory protein 1 |
| ZFYVE27 | Protrudin |
| SLC33A1 | Solute carrier family 33 (acetyl- CoA transporter) |
| CYP7B1 | Cytochrome P450, family 7, subfamily B, polypeptide 1 |
| SPG7 | Paraplegin |
| SPG11 | Spatacsin |
| ZFYVE26 | Spastizin |
| ERLIN2 | ER lipid raft associated 2 |
| SPG20 | Spartin |
| SPG21 | Maspardin |
| B4GALNT1 | beta-1,4-N-acetyl-galactosaminyl transferase 1 |
| DDHD1 | DDHD domain containing 1 |
| KIF1A | Kinesin family member 1A |
| FA2H | Fatty acid 2-hydroxylase |
| PNPLA6 | Patatin-like phospholipase domain containing 6 |
| C19orf12 | chromosome 19 open reading frame 12 |
| GJC2 | gap junction protein, gamma 2, 47 kDa |
| NT5C2 | 5′-nucleotidase, cytosolic II |
| GBA2 | glucosidase, beta (bile acid) 2 |
| AP4B1 | adaptor-related protein complex 4, beta 1 subunit |
| AP5Z1 | Hypothetical protein LOC9907 |
| TECPR2 | tectonin beta-propeller repeat containing 2 |
| AP4M1 | Adaptor-related protein complex 4, mu 1 subunit |
| AP4E1 | Adaptor-related protein complex 5, zeta 1 subunit |
| AP4S 1 | adaptor-related protein complex 4, sigma 1 subunit |
| DDHD2 | DDHD domain containing 2 |
| C12orf65 | adaptor-related protein complex 4, sigma 1 subunit |
| CYP2U1 | cytochrome P450, family 2, subfamily U, polypeptide 1 |
| ARL6IP1 | ADP-ribosylation factor-like 6 interacting protein 1 |
| AMPD2 | adenosine monophosphate deaminase 2 |
| ENTPD1 | ectonucleoside triphosphate diphosphohydrolase 1 |
| ALDH3A2 | Aldehyde dehydrogenase 3A2 |
| ALS2 | Alsin |
| L1CAM | L1 cell adhesion molecule |
| PLP1 | Proteolipid protein 1 |
| MTPAP | mitochondrial poly(A) polymerase |
| AFG3L2 | AFG3 ATPase family gene 3-like 2 (S. cerevisiae) 1 |
| SACS | Sacsin |

Other neuromuscular disorders

| | |
|---|---|
| Gene | protein |
| TOR1A | Torsin A |
| SGCE | Sarcoglycan, epsilon |
| IKBKAP | Inhibitor of kappa light polypeptide gene enhancer in B-cells, kinase complex-associated protein |
| TTR | Transthyretin (prealbumin, amyloidosis type I) |
| KIF21A | Kinesin family member 21A |
| PHOX2A | Paired-like aristaless homeobox protein 2A |
| TUBB3 | Tubulin, beta 3 |
| TPM2 | Tropomyosin 2 (beta) |
| MYH3 | Myosine, heavy chain 3, skeletal muscle, embryonic |
| TNNI2 | Troponin I, type 2 |
| TNNT3 | Troponin T3, skeletal |
| SYNE1 | Spectrin repeat containing, nuclear envelope 1 (nesprin 1) |
| MYH8 | Myosin heavy chain, 8, skeletal muscle, perinatal |
| POLG | Polymerase (DNA directed), gamma |
| SLC25A4 | Mitochondrial carrier; adenine nucleotide translocator |
| C10orf2 | chromosome 10 open reading frame 2 |
| POLG2 | Mitochondrial DNA polymerase, accessory subunit |
| RRM2B | Ribonucleotide reductase M2 B (TP53 inducible) |
| TK2 | Thymidine kinase 2, mitochondrial |
| SUCLA2 | Succinate-CoA ligase, ADP-forming, beta subunit |
| OPA1 | optic atrophy 1 |
| STIM1 | Stromal interaction molecule 1 |
| ORAI1 | ORAI calcium release-activated calcium modulator 1 |
| PUS1 | Pseudouridylate synthase 1 |
| CHCHD10 | Coiled-coil-helix-coiled-coil-helix domain containing 10 |
| CASQ1 | Calsequestrin 1 (fast-twitch, skeletal muscle) |
| YARS2 | tyrosyl-tRNA synthetase 2, mitochondrial |

Hereditary ataxia

| | |
|---|---|
| Gene symbol | protein |
| ATXN1 | Ataxin 1 |
| ATXN2 | Ataxin 2 |
| ATXN3 | Ataxin 3 |
| SPTBN2 | Spectrin, beta, non-erythrocytic 2 |
| CACNA1 A | Calcium channel, voltage-dependent, P/Q type, alpha 1A subunit |
| ATXN7 | Ataxin 7 |
| ATXN8OS | Ataxin 8 opposite strand |
| ATXN10 | Ataxin 10 |
| TTBK2 | Tau tubulin kinase 2 |
| PPP2R2B | Protein phosphatase 2 regulatory subunit B, beta isoform |
| KCNC3 | Potassium voltage-gated channel, Shaw-related subfamily, member 3 |
| PRKCG | Protein kinase C, gamma |
| ITPR1 | Inositol 1,4,5-triphosphate receptor type 1 |
| TBP | TATA box binding protein |
| IFRD1 | Interferon-related developmental regulator 1 |
| KCND3 | Potassium voltage-gated channel, Shal-related subfamily, member 3 |
| PDYN | prodynorphin |
| EEF2 | Eukaryotic translation elongation factor 2 |
| FGF14 | Fibroblast growth factor 14 |
| AFG3L2 | AFG3 ATPase family gene 3-like 2 (S. cerevisiae) 1 |
| BEAN1 | Brain expressed, associated with Nedd42 |
| TK2 | Thymidine kinase 2, mitochondrial |
| ELOVL4 | ELOVL fatty acid elongase 4 |
| TGM6 | Transglutaminase 6 |
| NOP56 | NOP56 ribonucleoprotein |
| ELOVL5 | ELOVL fatty acid elongase 5 |
| CCDC88C | Coiled-coil domain containing 88C |
| KCNA1 | Potassium voltage-gated channel, shaker-related subfamily, member 1 |
| CACNB4 | Calcium channel, voltage-dependent, beta 4 subunit |
| SLC1A3 | EAAT1 (excitatory amino acid transporter type 1) |
| FXN | Frataxin |
| TTPA | Tocopherol (alpha) transfer protein (ataxia (Friedreich-like) with vitamin E deficiency) |
| C10orf2 | chromosome 10 open reading frame 2 |
| APTX | Aprataxin |
| SETX | Senataxin |
| SYNE1 | Spectrin repeat containing, nuclear envelope 1 (nesprin 1) |
| ADCK3 | Atypical kinase ADCK3, mitochondrial |
| TDP1 | Tyrosyl-DNA phosphodiesterase 1 |
| SIL1 | SIL1 homolog, endoplasmic reticulum chaperone |
| POLG | Polymerase (DNA directed), gamma |
| ATM | Ataxia telangiectasia mutated |
| MRE11A | MRE11 meiotic recombination 11 homolog A |
| SACS | Sacsin |
| PHYH | Phytanoyl-CoA 2-hydroxylase |
| PEX7 | Peroxisomal biogenesis factor 7 |
| RNF216 | Ring finger protein 216 |

Any one of the above listed genes may be targeted in replacement gene therapy, wherein the gene of interest is a functional version of the deficient or mutated gene

Alternatively, the above listed genes may be used as target for gene editing. Gene editing is used to correct the sequence of a mutated gene or modify the expression or regulation of a deficient/abnormal gene so that a functional gene is expressed in muscle cells. In such cases, the gene of interest is chosen from those encoding therapeutic RNAs such as interfering RNAs, guide RNAs for genome editing and antisense RNAs capable of exon skipping, wherein the therapeutic RNAs target the preceding list of genes. Tools such as CRISPR/Cas9 may be used for that purpose.

Thus, by gene editing or gene replacement a correct version of this gene is provided in muscle cells and/or cells of the nervous system (PNS and/or CNS) of affected patients, in particular muscle cells and cells of the CNS of affected patients, this may contribute to effective therapies against this disease.

In some embodiments, the target gene for gene therapy (additive gene therapy or gene editing) is a gene responsible for one of the neuromuscular diseases listed above, preferably selected from the group comprising : (i) myopathies, such as hereditary cardiomyopathies, metabolic myopathies, other myopathies, distal myopathies, muscular dystrophies and congenital myopathies; (ii) spinal muscular atrophies (SMAs) and motor neuron diseases; (iii) Myotonic syndrome, in particular myotonic dystrophy type 1 and type 2; Congenital myasthenic syndromes; Hereditary motor and sensory neuropathies; Hereditary paraplegia and Hereditary ataxia, in particular, congenital myopathies and muscular dystrophies, and spinal muscular atrophies (SMAs) and motor neuron diseases.

In some particular embodiments, the target gene for gene therapy (additive gene therapy or gene editing) is a gene responsible for one of the neuromuscular diseases listed above, preferably selected from the group comprising Duchenne muscular dystrophy and Becker muscular dystrophy (DMD gene), Limb-girdle muscular dystrophies (LGMDs) (CAPN3, DYSF, FKRP, ANO5 genes and others), Spinal muscular atrophy (SMN1, ASAH1 genes) and Amyotrophic lateral sclerosis (SOD1, ALS2, SETX, FUS, ANG, TARDBP, FIG4, OPTN and others), Myotubular myopathy (MTM1 gene), Centronuclear myopathies (MTM1, DNM2, BIN1 genes), Nemaline myopathies (ACTA1, KLHL40, KLHL41, KBTBD13 genes), Selenoprotein N-related myopathy (SEPN1 gene), Congenital myasthenia (ColQ, CHRNE, RAPSN, DOK7, MUSK genes), Pompe disease (GAA gene), Glycogen storage disease III (GSD3) (AGL gene), Myotonic dystrophy type 1 (DMPK gene) and type 2 (CNBP/ZNF9 gene); Hereditary paraplegia (SPAST) and Charcot-Marie-Tooth, Type 4B1 (MTMR2). In some more preferred embodiments, the target gene is selected from the group consisting of : DMD, CAPN3, DYSF, FKRP, ANO5, MTM1, DNM2, BIN1, ACTA1, KLHL40, KLHL41, KBTBD13, TPM3, TPM2, TNNT1, CFL2, LMOD3, SEPN1, GAA, AGL, SMN1, and ASAH1 genes.

In some preferred embodiments, the target gene for gene therapy (additive gene therapy or gene editing) is a gene responsible for one of the neuromuscular diseases affecting at least the nervous system listed above, preferably selected from the group comprising : (i) myopathies, such as muscular dystrophies, including congenital muscular dystrophies; (ii) spinal muscular atrophies (SMAs) and motor neuron diseases; (iii) Myotonic syndrome, in particular myotonic dystrophy type 1 and type 2; (iv) Hereditary motor and sensory neuropathies; (v) Hereditary paraplegia and Hereditary ataxia; (vi) Congenital myasthenic syndromes, in particular muscular dystrophies including congenital muscular dystrophies, congenital myasthenic syndromes , and spinal muscular atrophies (SMAs) and motor neuron diseases.

In some more preferred embodiments, the target gene for gene therapy is a gene responsible for a myopathy affecting at least the nervous system, such as muscular dystrophy, including congenital muscular dystrophy affecting at least the nervous system, selected from the group consisting of: FKTN, POMT1, POMT2, POMGNT1, POMGNT2, LMNA, ISPD, GMPPB, LARGE, LAMA2, TRIM32, and B3GALNT2.

In some other more preferred embodiments, the target gene for gene therapy is a gene responsible for a myopathy affecting at least the nervous system, such as congenital myasthenic syndromes, e.g., Congenital myasthenia, selected from the genes responsible for congenital myasthenic syndromes listed in the Table described above.

In some other more preferred embodiments, the target gene for gene therapy (additive gene therapy or gene editing) is a gene responsible for one of the neuromuscular diseases affecting at least the nervous system listed above, preferably selected from the group comprising Duchenne muscular dystrophy and Becker muscular dystrophy (DMD gene), Limb-girdle muscular dystrophies (LGMDs) (DYSF, FKRP), Spinal muscular atrophy (SMN1, ASAH1 genes) and Amyotrophic lateral sclerosis (SOD1, ALS2, SETX, FUS, ANG, TARDBP, FIG4, OPTN and others), Centronuclear myopathies (DNM2, BIN1 genes), Pompe disease (GAA gene), Glycogen storage disease III (GSD3) (AGL gene), Myotonic dystrophy type 1 (DMPK gene) and type 2 (CNBP/ZNF9 gene); Hereditary paraplegia (SPAST (SPG4), SPG7, and other SPG genes such as SPG11, SPG20 and SPG21; in particular SPAST (SPG4) and SPG7); Charcot-Marie-Tooth, Type 4B1 (MTMR2); and Congenital myasthenic syndromes, e.g., Congenital myasthenia (CHAT, AGRN genes).

In some even more preferred embodiments, the target gene is selected from the group consisting of: DMD, DYSF, FKRP, DNM2, BIN1, GAA, AGL, SMN1, and ASAH1 genes.

In some preferred embodiments, the peptide-modified AAVpo1 vector according to the present disclosure is used to target motor neurons for treating motor neuron diseases. The target gene may be any one of the genes involved in Spinal muscular atrophies (SMAs) & Motor Neuron diseases listed in the Table above. Motor neuron diseases include amyotrophic lateral sclerosis (ALS), progressive bulbar palsy (PBP), pseudobulbar palsy, progressive muscular atrophy (PMA), primary lateral sclerosis (PLS), spinal muscular atrophy (SMA) and monomelic atrophy (MMA), as well as some rarer variants resembling ALS.

Dystrophinopathies are a spectrum of X-linked muscle diseases caused by pathogenic variants in DMD gene, which encodes the protein dystrophin. Dystrophinopathies comprises Duchenne muscular dystrophy (DMD), Becker muscular dystrophy (BMD) and DMD-associated dilated cardiomyopathy.

The Limb-girdle muscular dystrophies (LGMDs) are a group of disorders that are clinically similar to DMD but occur in both sexes as a result of autosomal recessive and autosomal dominant inheritance. Limb-girdle dystrophies are caused by mutation of genes that encode sarcoglycans and other proteins associated with the muscle cell membrane, which interact with dystrophin. The term LGMD1 refers to genetic types showing dominant inheritance (autosomal dominant), whereas LGMD2 refers to types with autosomal recessive inheritance. Pathogenic variants at more than 50 loci have been reported (LGMD1A to LGMD1G; LGMD2A to LGMD2W). Calpainopathy (LGMD2A) is caused by mutation of the gene CAPN3 with more than 450 pathogenic variants described. Contributing genes to LGMD phenotype include: anoctamin 5 (ANO5), blood vessel epicardial substance (BVES), calpain 3 (CAPN3), caveolin 3 (CAV3), CDP-L-ribitol pyrophosphorylase A (CRPPA), dystroglycan 1 (DAG1), desmin (DES), DnaJ heat shock protein family (Hsp40) homolog, subfamily B, member 6 (DNAJB6), dysferlin (DYSF), fukutin related protein (FKRP), fukutin (FKT), GDP-mannose pyrophosphorylase B (GMPPB), heterogeneous nuclear ribonucleoprotein D like (HNRNPDL), LIM zinc finger domain containing 2 (LIMS2), lain A:C (LMNA), myotilin (MYOT), plectin (PLEC), protein O-glucosyltransferase 1 (PLOGLUT1), protein O-linked mannose N-acetylglucosaminyltransferase 1 (beta 1,2-) (POMGNT1), protein O-mannose kinase (POMK), protein O-mannosyltransferase 1 (POMT1), protein O-mannosyltransferase 2 (POMT2), sarcoglycan alpha (SGCA), sarcoglycan beta (SGCB), sarcoglycan delta (SGCD), sarcoglycan gamma (SGCG), titin-cap (TCAP), transportin 3 (TNPO3), torsin 1A interacting protein (TOR1AIP1), trafficking protein particle complex 11 (TRAPPC11), tripartite motif containing 32 (TRIM 32) and titin (TTN). Major contributing genes to LGMD phenotype include CAPN3, DYSF, FKRP and ANO5 (Babi Ramesh Reddy Nallamilli et al., Annals of Clinical and Translational Neurology, 2018, 5, 1574-1587.

Dysferlin is involved in neurological disorders including multiple sclerosis (Hochmeister et al., J. Neuropathol. Exp. Neurol., 2006 Sep;65(9):855-65); Alzheimer (Galvin et al., Acta Neuropathol., 2006 Dec;112(6):665-71 and choreic movement (Takahashi T, et al., Mov. Disord., 2006, Sep;21(9):1513-5).

Spinal muscular atrophy is a genetic disorder caused by mutations in the Survival Motor Neuron 1 (SMN1) gene which is characterized by weakness and wasting (atrophy) in muscles used for movement. Mutations in ASAH1 gene lead to SMA-PME (spinal muscular atrophy with progressive myoclonic epilepsy).

X-linked myotubular myopathy is a genetic disorder caused by mutations in the myotubularin (MTM1) gene which affects muscles used for movement (skeletal muscles) and occurs almost exclusively in males. This condition is characterized by muscle weakness (myopathy) and decreased muscle tone (hypotonia).

Pompe disease is a genetic disorder caused by mutations in the acid alpha-glucosidase (GAA) gene. Mutations in the GAA gene prevent acid alpha-glucosidase from breaking down glycogen effectively, which allows this sugar to build up to toxic levels in lysosomes. This buildup damages organs and tissues throughout the body, particularly the muscles, leading to the progressive signs and symptoms of Pompe disease.

Glycogen storage disease III (GSD3) is an autosomal recessive metabolic disorder caused by homozygous or compound heterozygous mutation in the Amylo-Alpha-1, 6-Glucosidase, 4-Alpha-Glucanotransferase (AGL) gene which encodes the glycogen debrancher enzyme and associated with an accumulation of abnormal glycogen with short outer chains. Clinically, patients with GSD III present in infancy or early childhood with hepatomegaly, hypoglycemia, and growth retardation. Muscle weakness in those with IIIa is minimal in childhood but can become more severe in adults; some patients develop cardiomyopathy.

Genome-wide association studies identified the BIN1 locus as a leading modulator of genetic risk in Alzheimer’s disease (AD) (Voskobiynyk et al., eLife doi: 10.7554/eLife.57354; Jul. 13, 2020). Hereditary spastic paraplegias (HSPs) are a group of rare, inherited, neurological diseases characterized by broad clinical and genetic heterogeneity. Lower-limb spasticity with first motoneuron involvement is the core symptom of all HSPs. The genes responsible for HSPs include at least 79 SPG genes. Mutations in SPG7 and SPAST are common causes of hereditary spastic paraplegia (HSP) (Review in Lallemant-Dudek P. et al. Fac. Rev., 2021, Mar 10;10:27).

A non-limiting example of vector for use in the gene therapy of myotubular myopathy is a AAVpo1 vector comprising a peptide-modified capsid protein comprising the sequence SEQ ID NO: 5 or a sequence having at least 95%, 96% ,97%, 98% or 99% identity with said sequence which comprises the peptide of any one of SEQ ID NO : 2 to 4, said vector further packaging a human MTM1 gene operably linked to a human desmin promoter, and further operably linker to a miR208a target sequence. This vector is useful for expressing the gene of interest in skeletal muscles but not in the liver following systemic administration such as intravascular injection.

Another non-limiting example of vector for use in the gene therapy of spinal muscular atrophy is a AAVpo1 vector comprising a peptide-modified capsid protein comprising the sequence SEQ ID NO: 5 or a sequence having at least 95%, 96% , 97%, 98% or 99% identity with said sequence which comprises the peptide of any one of SEQ ID NO : 2 to 4, said vector further packaging a human SMN1 gene operably linked to a CAG promoter, and preferably further comprising human beta globin polyadenylation signal. This vector is useful for expressing the gene of interest in muscles including heart and in the nervous system, in particular in muscles including heart and in the CNS but not in the liver following systemic administration such as intravascular injection.

The pharmaceutical composition of the invention which comprises peptide-modified AAVpo1 vector particles with reduced liver tropism may be administered to patients having concurrent liver degeneration such as fibrosis, non-alcoholic fatty liver disease, non-alcoholic steatohepatitis, viral or toxic hepatitis or underlying genetic disorders inducing liver degeneration.

In the context of the invention, a therapeutically effective amount refers to a dose sufficient for reversing, alleviating or inhibiting the progress of the disorder or condition to which such term applies, or reversing, alleviating or inhibiting the progress of one or more symptoms of the disorder or condition to which such term applies.

The effective dose is determined and adjusted depending on factors such as the composition used, the route of administration, the physical characteristics of the individual under consideration such as sex, age and weight, concurrent medication, and other factors, that those skilled in the medical arts will recognize.

In the various embodiments of the present invention, the pharmaceutical composition comprises a pharmaceutically acceptable carrier and/or vehicle.

A “pharmaceutically acceptable carrier” refers to a vehicle that does not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Preferably, the pharmaceutical composition contains vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or suspensions. The solution or suspension may comprise additives which are compatible with viral vectors and do not prevent viral vector particle entry into target cells. In all cases, the form must be sterile and must be fluid to the extent that easy syringe ability exists. It must be stable under the conditions of manufacture and storage and must be preserved against the contaminating action of microorganisms, such as bacteria and fungi. An example of an appropriate solution is a buffer, such as phosphate buffered saline (PBS) or Ringer lactate.

The invention provides also a method for treating a muscle or nervous system disorder, in particular muscle or CNS disorder according to the present disclosure, comprising: administering to a patient a therapeutically effective amount of the pharmaceutical composition as described above. More preferably, the invention provides a method for treating a muscle and nervous system disorder, in particular muscle and CNS disorder according to the present disclosure.

The invention provides also the use of the pharmaceutical composition according to the present disclosure for the preparation of a medicament for treating a muscle or nervous system disorder, in particular muscle or CNS disorder according to the present disclosure; preferably a muscle and nervous system disorder, in particular muscle and CNS disorder according to the present disclosure.

As used herein, the term “patient” or “individual” denotes a mammal. Preferably, a patient or individual according to the invention is a human.

In the context of the invention, the term “treating” or “treatment”, as used herein, means reversing, alleviating or inhibiting the progress of the disorder or condition to which such term applies, or reversing, alleviating or inhibiting the progress of one or more symptoms of the disorder or condition to which such term applies.

The pharmaceutical composition of the present invention, is generally administered according to known procedures, at dosages and for periods of time effective to induce a therapeutic effect in the patient.

The administration can be systemic, local or systemic combined with local. Systemic administration is preferably parenteral such as subcutaneous (SC), intramuscular (IM), intravascular such as intravenous (IV) or intraarterial; intraperitoneal (IP); intradermal (ID) or else. Local administration is preferably intracerebral, intracerebroventricular, intracisternal, and/or intrathecal administration. The administration may be for example by injection or perfusion. In some preferred embodiments, the administration is parenteral, preferably intravascular such as intravenous (IV) or intraarterial. In some other preferred embodiments, the administration is intracerebral, intracerebroventricular, intracisternal, and/or intrathecal administration, alone or combined with parenteral administration, preferably intravascular administration. In some other preferred embodiments, the administration is parenteral, preferably intravascular alone or combined with intracerebral, intracerebroventricular, intracisternal, and/or intrathecal administration

The practice of the present invention will employ, unless otherwise indicated, conventional techniques, which are within the skill of the art. Such techniques are explained fully in the literature.

The invention will now be exemplified with the following examples, which are not limitative, with reference to the attached drawings in which:

### FIGURE LEGENDS

FIG. 1: Body weight over time of Mtml-KO mice treated with various AAV vectors expressing hMTM1. AAVpo1 (KO-AAVpo1), AAVpo1A1 (KO-AAVpo1A1), AAV8 (KO-AAV8), AAV9 (KO-AAV9), AAVrh10 (KO-AAVrh10). Untreated wild-type (WT-PBS) and Mtml-KO (KO-PBS) mice are used as controls.

FIG. 2: Muscles weight of Mtml-KO mice treated with various AAV vectors expressing hMTM1. AAVpo1 (KO + AAVpo1), AAVpo1A1 (KO + AAVpo1A1), AAV8 (KO + AAV8), AAV9 (KO + AAV9), AAVrh10 (KO + AAVrh10). Untreated wild-type (WT + PBS) and Mtml-KO (KO + PBS) mice are used as controls. TA: Tibialis Anterior EDL: Extensor Digitorum Longus. Qua: Quadriceps. Ga: Gastrocnemius. Sol: Soleus. Triceps. Biceps. Diaphragm. Heart. Statistical analyses were performed using one-way ANOVA followed by Tukey’s multiple comparison post-test (* P<0.05 vs. KO + AAV8; ** P<0.01 vs. KO + AAV; *** P<0.001 vs. KO + AAV8; $ P<0.05 vs. KO + AAV9; $$ P<0.01 vs. KO + AAV9; $$$ P<0.001 vs. KO + AAV9).

FIG. 3: Vector Copy Number (VCN) in muscles of Mtml-KO mice treated with various AAV vectors expressing hMTM1. AAVpo1 (KO + AAVpo1), AAVpo1A1 (KO + AAVpo1A1), AAV8 (KO + AAV8), AAV9 (KO + AAV9), AAVrh10 (KO + AAVrh10). Untreated wild-type mice (WT-PBS) are used as control. TA: Tibialis Anterior EDL: Extensor Digitorum Longus. Qua: Quadriceps. Ga: Gastrocnemius. Sol: Soleus. Triceps. Biceps. Diaphragm. Heart. Statistical analyses were performed using one-way ANOVA followed by Tukey’s multiple comparison post-test (* P<0.05 vs. KO + AAV8; ** P<0.01 vs. KO + AAV; *** P<0.001 vs. KO + AAV8; $ P<0.05 vs. KO + AAV9; $$ P<0.01 vs. KO + AAV9).

FIG. 4: Vector Copy Number (VCN) in organs of Mtml-KO mice treated with various AAV vectors expressing hMTM1. AAVpo1 (KO + AAVpo1), AAVpo1A1 (KO + AAVpo1A1), AAV8 (KO + AAV8), AAV9 (KO + AAV9), AAVrh10 (KO + AAVrh10). Statistical analyses were performed using one-way ANOVA followed by Tukey’s multiple comparison post-test (* P<0.05 vs. KO + AAV8; ** P<0.01 vs. KO + AAV; *** P<0.001 vs. KO + AAV8; $ P<0.05 vs. KO + AAV9; $$ P<0.01 vs. KO + AAV9; $$$ P<0.001 vs. KO + AAV9).

FIG. 5: hMTM1 mRNA level in muscles of Mtml-KO mice treated with various AAV vectors expressing hMTM1. AAVpo1 (KO + AAVpo1), AAVpo1A1 (KO + AAVpo1A1), AAV8 (KO + AAV8), AAV9 (KO + AAV9), AAVrh10 (KO + AAVrh10). MTM1 mRNA levels are expressed relative to expression in KO + AAV8. TA: Tibialis EDL: Extensor Digitorum Anterior Longus. Qua: Quadriceps. Ga: Gastrocnemius. Sol: Soleus. Triceps. Biceps. Diaphragm. Heart. Statistical analyses were performed by using one-way ANOVA followed by Tukey’s multiple comparison post-test (* P<0.05 vs. KO + AAV8; ** P<0.01 vs. KO + AAV; *** P<0.001 vs. KO + AAV8; $ P<0.05 vs. KO + AAV9; $$ P<0.01 vs. KO + AAV9; $$$ P<0.001 vs. KO + AAV9).

FIG. 6: hMTM1 mRNA level in organs of Mtml-KO mice treated with various AAV vectors expressing MTM1. AAVpo1 (KO + AAVpo1), AAVpo1A1 (KO + AAVpo1A1), AAV8 (KO + AAV8), AAV9 (KO + AAV9), AAVrh10 (KO + AAVrh10). hMTM1 mRNA levels are expressed relative to expression in KO + AAV8. Statistical analyses were performed using one-way ANOVA followed by Tukey’s multiple comparison post-test (* P<0.05 vs. KO + AAV8; ** P<0.01 vs. KO + AAV; *** P<0.001 vs. KO + AAV8; $ P<0.05 vs. KO + AAV9; $$$ P<0.001 vs. KO + AAV9).

FIG. 7: hMTM1 protein level in muscles of Mtml-KO mice treated with various AAV vectors expressing hMTM1. AAVpo1 (KO + AAVpo1), AAVpolAl (KO + AAVpo1A1), AAV8 (KO + AAV8), AAV9 (KO + AAV9), AAVrh10 (KO + AAVrh10). Untreated wild-type (WT + PBS) and Mtml-KO (KO + PBS) mice are used as controls. GAPDH is used as internal control.

FIG. 8: Immunolocalization of SMN protein in neurons of spinal cord of C57BL/6 mice injected with AAVpo1A1-SMN1 vector at 5x10¹³ vg/kg. The SMN protein is fused to an HA-tag and detected with an anti-HA antibody. Neurons were labelled with an anti-NeuN antibody. Arrows show motoneurons expressing HA-SMN. Scale bars = 200 µm (left) or 50 µm (right).

### EXAMPLES

### Materials and Methods

The AAVpo1A1 capsid of porcine origin (nucleotide sequence SEQ ID NO: 13 encoding the protein of SEQ ID NO: 5 comprising the peptide of SEQ ID NO: 4 replacing all the residues from positions 567-569 and 570-572 of AAVpo1 capsid protein of SEQ ID NO: 1) was compared with the serotypes 8, 9, rh10 and po1 (Bello et al., Gene Therapy, 2009, 16, 1320-1328. doi: 10.1038/gt.2009.821), in a constitutive knockout of the myotubularin gene (Mtm1 KO mouse line) described previously (Buj-Bello et al., PNAS, 2002, 99, 15060-5. doi:10.1073/pnas.212498399; Al-Qusairi, et al., PNAS, 2009, 106, 18763-8. doi:10.1073/pnas.0900705106). The vectors were all produced by a triple transfection method using HEK 293 cells and carried a cassette expressing human MTM1 under the control of the human desmin promoter (1 kb) and a target sequence of miR208a (Raguz et al., Dev. Biol., 1998, 201, 26-42; Paulin D & Li Z, Exp. Cell. Res., 2004, Nov 15;301(1):1-7; Roudault et al., Circulation, 2013, 128, 1094-104. doi: 10.1161/CIRCULATIONAHA.113.001340). The AAVpo1A1 and AAV9 capsids were also assessed in C57BL/6 mice, with a cassette expressing human SMN fused to an HA tag sequence under the control of the ubiquitous CAG promoter (Meyer et al., Molecular Therapy, 2015, 23. doi: 10.1038/mt.2014.210).

A single dose of 2x10¹³ vg/kg of each vector expressing MTM1 was administrated intravenously in 3-week-old mutant mice and tissues were harvested and frozen in nitrogen 4 weeks post-injection. As control, PBS was injected in Mtm1-KO and wild-type littermate males. C57BL/6 mice received a dose of 8x10¹² vg/kg of either AAV9 or AAVpo1A1 vectors at the age of 4 weeks, and tissues were collected 3 weeks later.

The number of vector genomes per diploid genome was quantified from 32 ng of total DNA by Taqman real-time PCR using a LightCycler480 thermocycler (Roche). The titin gene was used for standardization with primers and probe: 5′-AAAACGAGCAGTGACGTGAGC-3′ (forward; SEQ ID NO: 6), 5′-TTCAGTCATGCTGCTAGCGC-3′ (reverse; SEQ ID NO: 7) and 5′-TGCACGGAAGCGTCTCGTCTCAGTC-3′ (probe; SEQ ID NO: 8). Primers used for vector genome (MTMI) amplification were: 5′-TTGGTTGTCCAGTTTGGAGTCTACT-3′ (forward; SEQ ID NO: 9), 5′-CCGTCACTGCAATGCACAAG-3′ (reverse; SEQ ID NO: 10) and 5′-ATATCAAGCTCGTTTTGAC-3′ (probe; SEQ ID NO: 11). Primers used for vector genome (SMN1) amplification were: 5′-CAGTGCAGGCTGCCTATCAG-3′ (forward; SEQ ID NO: 15), 5′-TGTGGGCCAGGGCATTAG-3′ (reverse; SEQ ID NO: 16), 5′-AAGTGGTGGCTGGTGTG-3′ (probe; SEQ ID NO: 17). Other primers used for vector genome (SMN1) amplification were: 5′-GCTGCCTCCATTTCCTTCTG-3′ (forward; SEQ ID NO: 18), 5′-ACATACTTCCCAAAGCATCAGCAT-3′ (reverse; SEQ ID NO: 19), 5′-CACCACCTCCCATATGTCCAGATTCTCTTG-3′ (probe; SEQ ID NO: 20).

The level of MTM1 transcripts was quantified from 350 ng of total RNA subjected to reverse transcription using RevertAid H Minus Reverse Transcriptase kit (Thermo Scientific). Next, a cDNA amount was amplified by qPCR using a LightCycler480 thermocycler (Roche). The RPLP0 gene was used for standardization with primers and probe: 5′-CTCTGGAGAAACTGCTGCCT-3′ (forward; SEQ ID NO: 21), 5′-CTGCACATCACTCAGAATTTCAA-3′ (reverse; SEQ ID NO: 22) and 5′-AGGACCTCACTGAGATTCGGGATATGC -3′ (probe; SEQ ID NO: 23).

Proteins were extracted and analyzed by NuPAGE 4-12% Bis-Tris gel electrophoresis and western blotting. Membranes were probed with a polyclonal antibody against human myotubularin (Abnova). A mouse monoclonal antibody specific for GAPDH (Merck Millopore) was used as internal control. Detection was performed with a secondary antibody (Donkey anti-Goat 800 or Goat anti-Mouse 680 (Invitrogen) and the Odyssey infrared imaging system (LI-COR Biotechnology Inc.).

For immunostaining of the vector-derived HA-SMN, C57BL/6 mice were injected with a single dose of 5x10¹³ vg/kg of AAVpo1A1 vector and euthanized 4 weeks later by intraperitoneal anesthetic injection (10 mg/kg xylazine, 100 mg/kg ketamine), followed by intracardiac perfusion with PBS and then 4% paraformaldehyde (PFA). Tissues were isolated and postfixed by incubation in 4% PFA. Spinal cord was then incubated in a PBS-sucrose solution (30%). Serial coronal cryostat sections of lumbar spinal cord were processed for mouse IgG blocking with a Mouse-on-Mouse IgG Blocking Solution (Invitrogen), then anti-HA tag (hSMN) staining with a rabbit anti-HA primary antibody (Sigma-Aldrich) and for anti-NeuN staining with a mouse anti-NeuN primary antibody (Sigma-Aldrich). Detection was performed with fluorescent-conjugated secondary antibodies (Goat anti-rabbit Alexa Fluor 488 and Goat anti-mouse Alexa Fluor 594 (Invitrogen)). Sections were mounted with FluoroMount-G medium + DAPI, and the images were captured using the axioscan Z1 (Zeiss).

### Results

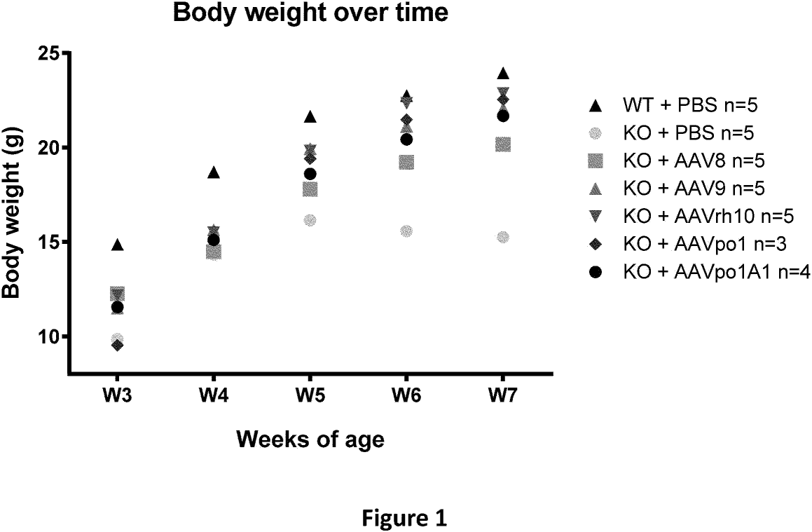

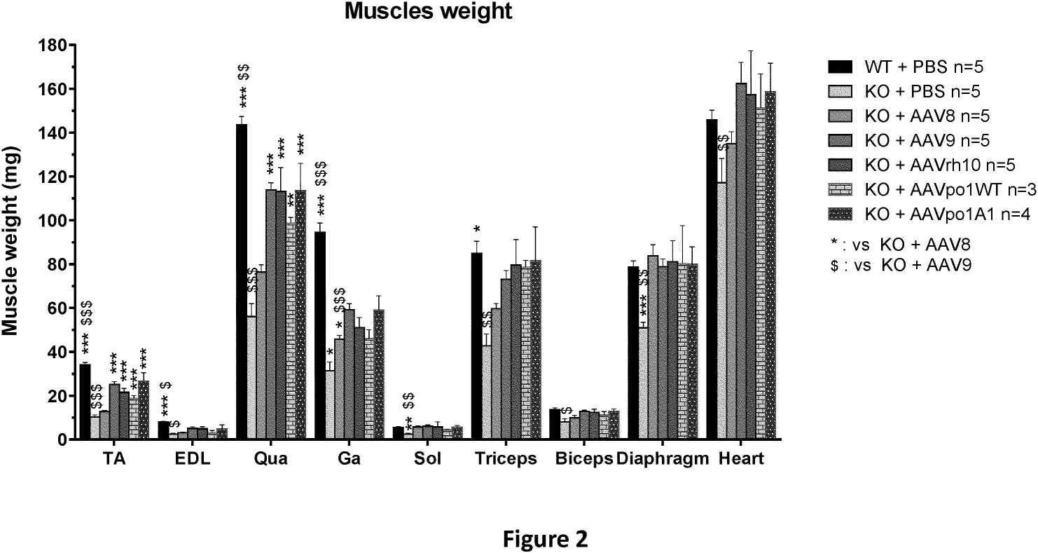

AAV vectors (AAVpo1, AAVpo1A1, AAV8, AAV9, AAVrh10) expressing MTM1 were injected intravenously at 2x10¹³ vg/kg in Mtm1-KO mice at 3 weeks of age. From two weeks post-injection, the body weight of treated KO and WT mice was similar, whereas untreated KO mice started to lose weight after 6 weeks of age (FIG. 1). Skeletal muscles of mutant mice from AAVpo1- and AAVpo1A1- treated groups, such as Tibialis Anterior (TA), quadriceps (Qua), gastrocnemius (Ga) and triceps (Tri), were heavier than in the AAV8-treated group mice (FIG. 2).

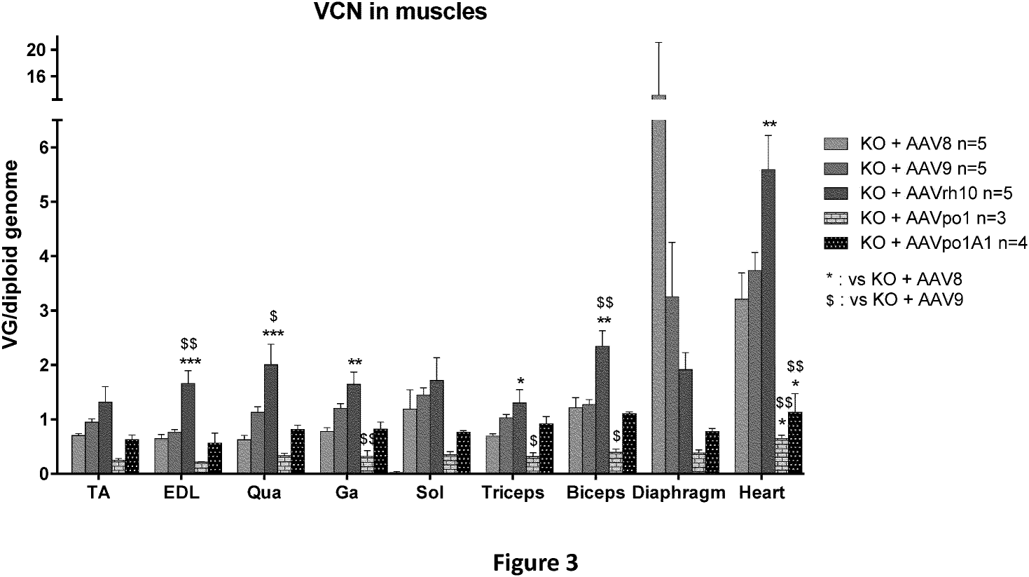

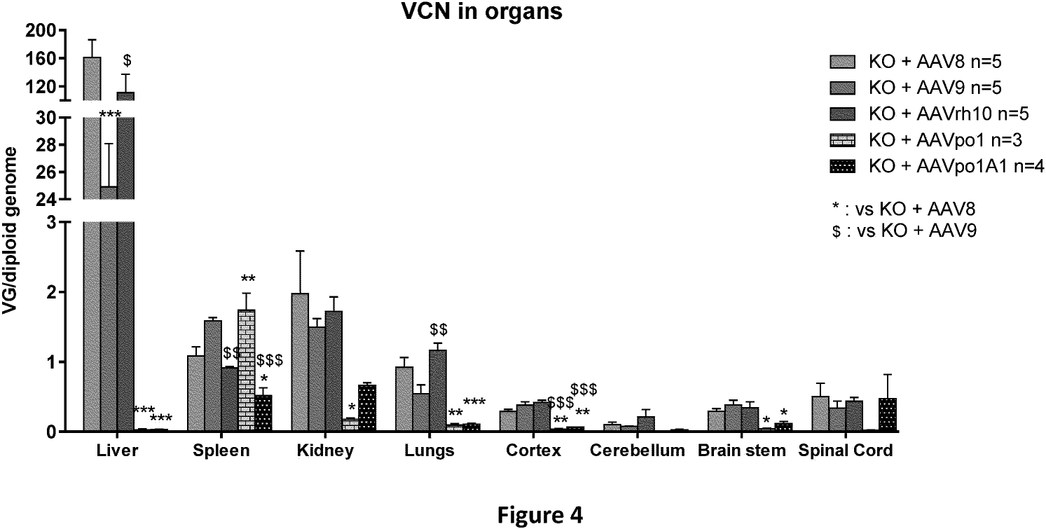

According to vector genome quantification in skeletal muscles (FIGS. 3 and 4), the AAVpo1A1 vector transduced most skeletal muscles as efficiently as the AAV8 vector. Interestingly, the AAVpo1A1 vector resulted in low transduction levels of organs such as heart, liver, spleen, kidney, lungs and brain.

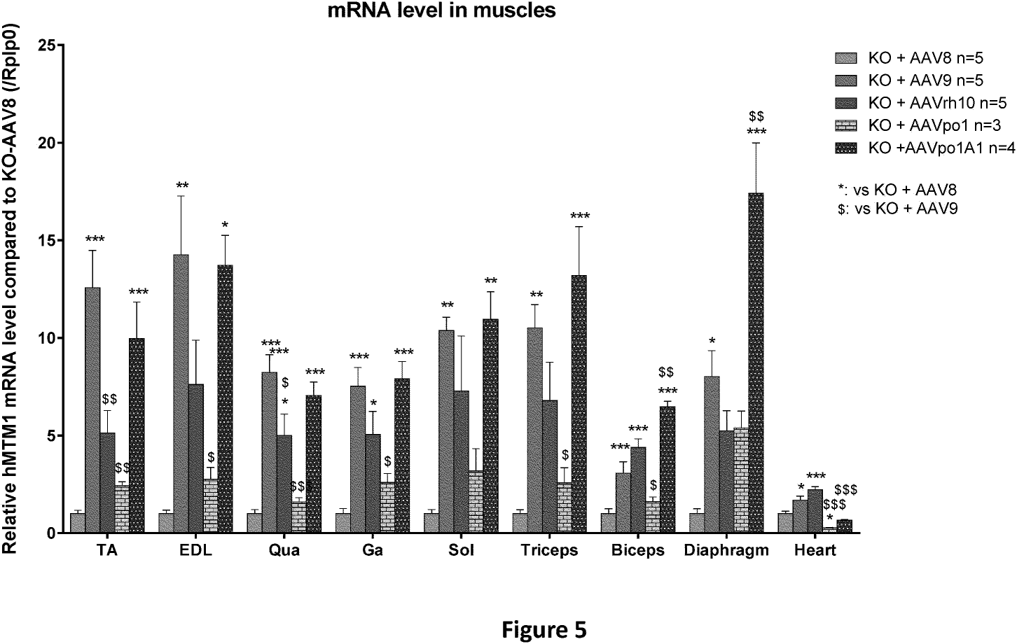

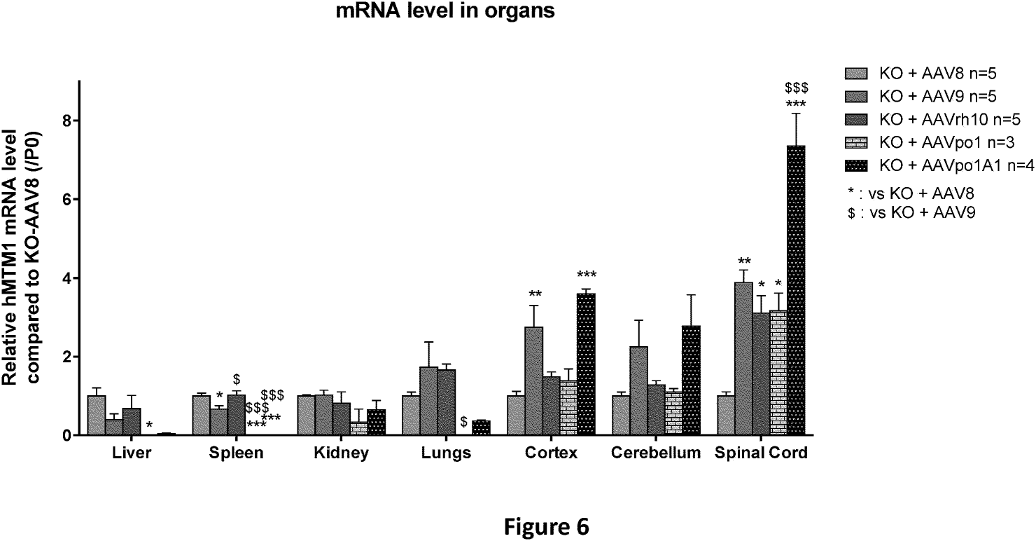

The expression of the MTM1 transgene was analyzed by RT-qPCR in various muscles and organs (FIGS. 5 and 6). The AAVpo1A1 vector led to higher MTM1 transcript levels in all skeletal muscles compared to AAV8, despite similar transduction levels, reaching transgene expression levels comparable to the AAV9 vector. Moreover, administration of the AAVpo1A1 vector detargeted transgene expression in organs such as liver and spleen, with much lower MTM1 transcript levels than those observed after AAV8 vector delivery. Transgene expression levels were higher in regions of the central nervous system, such as cortex and spinal cord, of AAVpo1A1-treated mice compared to the AAV8 group, and even higher than in AAV9-treated mice in the spinal cord.

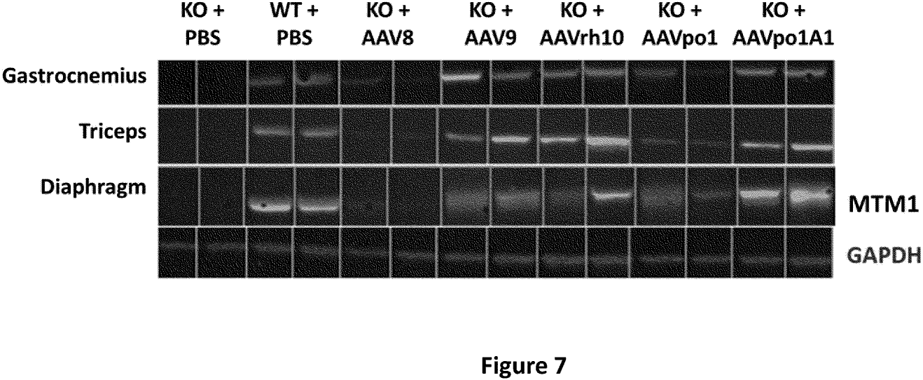

MTM1 protein expression was analyzed in various muscles (gastrocnemius, triceps, and diaphragm) by immunoblotting (FIG. 7). The AAVpo1A1 vector administration resulted in higher MTM1 protein levels in skeletal muscles of mutant mice compared to the AAV8 and AAVpo1 vectors.

The AAVpo1A1 and AAV9 vectors expressing SMN1 were injected intravenously at 8x10¹² vg/kg in C57BL/6 mice at 4 weeks of age. Several muscles and organs were collected 3 weeks later. The AAVpolAl vector transduced at similar levels all skeletal muscles and heart in WT mice. As previously observed in Mtm1-KO mice, administration of the AAVpo1A1 vector resulted in low transduction of the liver.

Transgene expression was analyzed by RT-qPCR, and results show that SMN1 transcript levels were similar in skeletal muscles after AAVpo1A1 and AAV9 vector transduction.. The levels of AAVpo1A1-derived SMN1 mRNA were lower in heart, liver, spleen and kidney compared to AAV9. In the central nervous system, AAVpo1A1-derived SMN1 transcripts were present in all analyzed regions (cortex, cerebellum and spinal cord), with levels slightly higher in spinal cord.

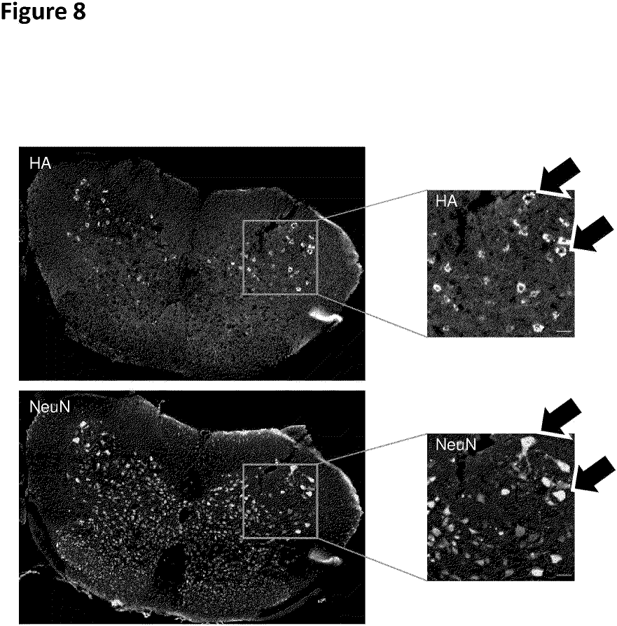

To assess the cellular localization of SMN in spinal cord, immunofluorescence stainings using anti-HA antibodies and anti-NeuN antibodies were performed 4 weeks after injection of AAVpo1A1-SMNI vector at 5x10¹³ vg/kg. As shown in FIG. 8, HA-SMN was expressed in neurons, and in particular in the large motoneurons located in the ventral horns of the spinal cord. The majority of motoneurons (mean 80%, range 72% to 94%, n=4 mice) were transduced with AAVpo1A1 and expressed the transgene.

Altogether this demonstrates the improved potency and tissue specificity of AAVpo1A1 vector for muscle- and/or CNS-directed gene transfer since it advantageously combines high transgene expression levels in skeletal muscle, brain and spinal cord comparable to the AAV9 vector and vector detargeted transgene expression in other organs such as liver and spleen.

1-16. (canceled)

17. A method of treating nervous system disorders and neuromuscular disorders affecting the nervous system by gene therapy in a patient in need thereof, comprising administering to the patient a therapeutically effective amount of a recombinant porcine adeno-associated virus (AAV) vector comprising a peptide-modified capsid protein,
wherein the peptide-modified capsid protein comprises at least one peptide comprising the sequence MPLGAAG (SEQ ID NO: 2) or a variant comprising only one or two amino acid mutations in said sequence, and
wherein said at least one peptide is inserted into a capsid from a porcine AAV serotype 1.

18. The method according to claim 17, wherein the recombinant porcine AAV vector is characterized by the combination of liver detargeting and transgene expression levels in different muscle groups, and in the brain and spinal cord that are at least equivalent if not superior to that of AAV9 vector, after systemic administration, in particular intravenous administration.

19. The method according to claim 17, wherein the peptide comprises the sequence GMPLGAAGA (SEQ ID NO: 3), or a variant comprising one or two amino acid deletions or substitutions in said sequence.

20. The method according to claim 19, wherein the peptide comprises or consists of the sequence GQRGMPLGAAGAQAA (SEQ ID NO: 4).

21. The method according to claim 17, wherein the peptide is inserted between residues N567 and S568 or between residues N569 and T570 of the capsid protein; said positions being determined by alignment with SEQ ID NO: 1.

22. The method according to claim 21, wherein the peptide replaces all the residues from positions 565-567 and 568-570 or all the residues from positions from positions 567-569 and 570-572; said positions being determined by alignment with SEQ ID NO: 1.

23. The method according to claim 17, wherein said peptide-modified capsid protein comprises a sequence selected from the group consisting of the sequence SEQ ID NO: 5, and the sequences having at least 95%, 96%, 97%, 98% or 99% identity with SEQ ID NO: 5 which comprise said peptide, and the fragment thereof corresponding to VP2 or VP3 capsid protein.

24. The method according to claim 17, wherein the recombinant porcine AAV vector is a vector particle packaging a gene of interest for therapy.

25. The method according to claim 24, wherein the gene of interest for therapy is operably linked to a promoter functional in neurons and/or glial cells.

26. The method according to claim 24, wherein the gene of interest for therapy is selected from the group consisting of:
(i) therapeutic genes;
(ii) genes encoding therapeutic proteins or peptides such as therapeutic antibodies or antibody fragments and genome editing enzymes; and
(iii) genes encoding therapeutic RNAs such as interfering RNAs, guide RNAs for genome editing and antisense RNAs capable of exon skipping.

27. The method according to claim 17, which is for treating a neuromuscular disease affecting the nervous system,.

28. The method according to claim 17, wherein the disease is a genetic neuromuscular disease affecting the nervous system.

29. The method according to claim 17, , wherein the disease is a genetic neuromuscular disease affecting the nervous system selected from the group comprising : (i) myopathies; (ii) spinal muscular atrophies and motor neuron diseases; (iii) Myotonic syndrome; (iv) Hereditary motor and sensory neuropathies; (v) Hereditary paraplegia and Hereditary ataxia; and (vi) Congenital myasthenic syndromes.

30. The method according to claim 29, wherein the myopathies are muscular dystrophies including congenital muscular dystrophies and/or the Myotonic syndrome is myotonic dystrophy type 1 or type 2.

31. The method according to claim 17, wherein the recombinant porcine AAV vector is a vector particle packaging a functional version of a gene responsible for a genetic neuromuscular disorder affecting the nervous system or a therapeutic RNA targeting said gene responsible for the disease.

32. The method according to claim 31, wherein the genetic neuromuscular disorder affecting the nervous system and the gene responsible for said disease are selected from the group comprising: Duchenne muscular dystrophy and Becker muscular dystrophy (DMD gene); Limb-girdle muscular dystrophies (DYSF, FKRP genes); Myotonic dystrophy type 1 (DMPK gene) and type 2 (CNBP/ZNF9 gene); Centronuclear myopathies (DNM2, BIN1 genes); Pompe disease (GAA gene); Glycogen storage disease III (AGL gene); Spinal muscular atrophy (SMN1, ASAH1 genes); Amyotrophic lateral sclerosis (SOD1, ALS2, SETX, FUS, ANG, TARDBP, FIG4, OPTN and others); Hereditary paraplegia (SPAST, SPG7); Charcot-Marie-Tooth, Type 4B1 (MTMR2), and Congenital myasthenic syndrome (CHAT, AGRN).

33. The method according to claim 31, wherein said gene responsible for the genetic neuromuscular disorder affecting the nervous system is selected from the group comprising: DMD, DYSF, FKRP, DNM2, BIN1, GAA, AGL, SMN1 and ASAH1 genes.

34. The method according to claim 31, wherein said gene responsible for the genetic neuromuscular disorder affecting the nervous system is selected from the group comprising: FKTN, POMT1, POMT2, POMGNT1, POMGNT2, LMNA, ISPD, GMPPB, LARGE, LAMA2, TRIM32, and B3GALNT2.

35. The method according to claim 17, which is for the gene therapy of Spinal muscular atrophy, wherein said vector comprises a peptide-modified capsid protein comprising the sequence SEQ ID NO: 5 or a sequence having at least 95%, 96%, 97%, 98% or 99% identity with said sequence which comprises the peptide of any one of SEQ ID NO: 2 to 4, and said vector further packaging a human SMN1 gene operably linked to a promoter functional in neurons and/or glial cells.

36. The method according to claim 17, wherein the recombinant porcine AAV vector is administered by systematic route, by intracerebral, intracerebroventricular, intracisternal, and/or intrathecal routes, or by a combination thereof.

37. The method according to claim 36, wherein the systemic route is an intravascular route.
